(19) United States
(12) Patent Application Publication
(10) Pub. No.: US 2025/0210137 A1
(43) Pub. Date: Jun. 26, 2025

(54) DIRECTLY DETERMINING SIGNAL-TO-NOISE-RATIO METRICS FOR ACCELERATED CONVERGENCE IN DETERMINING NUCLEOTIDE-BASE CALLS AND BASE-CALL QUALITY

(71) Applicant: Illumina, Inc., San Diego, CA (US)

(72) Inventors: Shu Xu, San Diego, CA (US); Rami Mehio, San Diego, CA (US); Robert Ezra Langlois, San Diego, CA (US); Eric Jon Ojard, San Francisco, CA (US)

(21) Appl. No.: 18/988,250

(22) Filed: Dec. 19, 2024

Related U.S. Application Data
(60) Provisional application No. 63/636,944, filed on Apr. 22, 2024.
(60) Provisional application No. 63/612,431, filed on Dec. 20, 2023.

(51) Int. Cl. G16B 30/00

(52) U.S. Cl. CPC G16B 30/00

(57) ABSTRACT

This disclosure describes methods, non-transitory computer readable media, and systems that generate an improved signal-to-noise-ratio metric for light signals emitted from fluorescent tags of nucleotide bases during a sequence run and use such signal-to-noise-ratio metrics to determine more accurate and flexible base calls. For instance, the disclosed systems can detect a series of signals from labeled nucleotide bases of a nucleotide-sample slide, determine intensity correction parameters based on intensity values for a given sequencing cycle, determine a scaling factor and a noise level based on the intensity correction parameters, and generate a signal-to-noise-ratio metric for the given sequencing cycle based on the scaling factor and the noise level. The disclosed systems can further utilize the signal-to-noise-ratio to generate quality metrics for generated nucleobase calls.

### CROSS-REFERENCE TO RELATED APPLICATIONS

This application claims priority to and the benefit of U.S. Provisional Patent Application No. 63/636,944, entitled, “DIRECTLY DETERMINING SIGNAL-TO-NOISE-RATIO METRICS FOR ACCELERATED CONVERGENCE IN DETERMINING NUCLEOTIDE-BASE CALLS AND BASE-CALL QUALITY,” filed on Apr. 22, 2024 and U.S. Provisional Patent Application No. 63/612,431, entitled, “SIGNAL-TO-NOISE RATIO METRIC FOR DETERMINING NUCLEOTIDE-BASE CALLS AND BASE-CALL QUALITY,” filed on Dec. 20, 2023. Both of the aforementioned applications are hereby incorporated by reference in its entirety.

### BACKGROUND

In recent years, biotechnology firms and research institutions have improved hardware and software platforms used for determining a sequence of nucleotide bases (also referred to as “nucleobases”) in a sample. For instance, some existing sequencing machines and sequencing-data-analysis software (together “existing sequencing systems”) determine individual nucleobases of nucleic-acid sequences by using conventional Sanger sequencing or by using sequencing-by-synthesis (SBS). When using SBS, existing sequencing systems can monitor thousands, tens of thousands, or more nucleic-acid polymers being synthesized in parallel to detect more accurate nucleobase calls. For instance, a camera in SBS platforms can capture images of irradiated fluorescent tags from nucleotide bases incorporated into such synthesized nucleic-acid sequences (often grouped into clusters of oligonucleotides). After capturing the images, a computing device from the existing systems uses sequencing-data-analysis software to determine nucleobases that were detected in a given image based on the light signal (e.g., the corresponding intensity values) captured in the image data. By iteratively incorporating nucleobases into the oligonucleotides and capturing images of the emitted light signals in various sequencing cycles, existing sequencing systems can determine the sequence of nucleobases present in the samples of nucleic acid.

Despite these recent advances, existing sequencing systems typically suffer from technical limitations that impede the accuracy of those systems. Many existing sequencing systems, for example, determine a variety of parameters for the correction of intensity values and the evaluation of base-calling quality determined during the sequencing process, such as a signal-to-noise-ratio (SNR) metric representing the strength of a given light signal detected from labeled nucleotide bases in comparison with a measure of noise associated with the given signal. Existing systems often determine many of these parameters using maximum likelihood (ML) model estimations that account for various attributes of the intensity values. Such systems, however, typically determine the SNR metrics using values obtained outside these ML model estimations at the current cycle. Indeed, some existing systems indirectly obtain the SNR metrics using a multi-step process that involves deriving one or more outside values based on the attributes of the intensity values and then determining the SNR metrics based on these outside values. Moreover, some existing sequencing systems determine SNR metrics for a target sequencing cycle based at least in part on a rolling average of noise levels (e.g., corrected intensity values) from previous cycles in the respective sequencing run.

As SNR metrics are key to evaluating base-calling quality, underestimation of SNR in these existing systems can lead to poor quality scores. To illustrate, existing systems that indirectly determine SNR metrics generally suffer from delayed convergence of the SNR metrics in early cycles of a sequencing run (e.g., due to inaccuracies or errors in the early cycles). As such, the SNR metrics determined during these early cycles are often unreliable and lead to poor quality scores. Some existing systems attempt to accommodate this delayed convergence by treating early-cycle SNR metrics differently than later-cycle SNR metrics (e.g., by weighting the early-cycle SNR metrics differently or omitting the early-cycle SNR metrics entirely during base call quality scoring). This approach, however, often leads to poor and inconsistent results. Further, by using a rolling average approach, existing systems typically propagate the errors associated with SNR metrics determined for earlier sequencing cycles to the SNR metrics determined for later cycles.

The inaccuracies of SNR metrics determined by many existing systems leads to inaccurate and/or low-quality scores for resultant nucleobase calls and further compromises to both primary and secondary procedures. Indeed, existing systems often rely on determined SNR metrics (e.g., the intensity value corrections enabled by the SNR metrics) to perform various functions, such as base calling, filtering out light signals from base call data (e.g., where the noise is too high), determining the quality of base calls, and/or generating or calibrating quality reference tables for quality scoring. By failing to accurately determine SNR metrics, existing systems degrade the performance of these other functions.

These, along with additional problems and issues exist in existing sequencing systems.

### SUMMARY

This disclosure describes embodiments of methods, non-transitory computer-readable media, and systems that determine improved signal-to-noise-ratio metrics for light signals emitted from fluorescent tags of nucleotide bases during a sequencing run. For example, the disclosed systems can determine a separate signal-to-noise-ratio metric for various clusters of oligonucleotides to which tagged nucleotide bases are added. In particular, the disclosed systems can implement an improved method for determining per-cluster signal-to-noise-ratio metrics across successive sequencing cycles directly from corresponding channel estimations. Indeed, in at least some cases, the improved method implemented by the disclosed systems provides for accelerated convergence of per-cluster signal-to-noise-ratio metrics during early sequencing cycles by calculating the per-cluster signal-to-noise-ratio metrics based directly on maximum-likelihood model estimates (e.g., of intensity correction parameters utilized to adjust intensity values of sequencing signals) for the respective sequencing cycle. In this manner, the disclosed systems can more accurately determine signal-to-noise-ratio metrics for clusters of oligonucleotides—particularly for early sequencing cycles.

The disclosed systems can utilize such signal-to-noise-ratio metrics associated with the clusters for a variety of base-calling applications. For example, the disclosed systems can use such signal-to-noise-ratio metrics to generate intensity-value boundaries for differentiating signals corresponding to different nucleotide bases according to a base-call-distribution model (e.g., segmented Gaussian mixture model), filter out clusters of poor quality, and/or determine a quality metric (e.g., a quality score) for nucleobase calls. Also, the disclosed systems can utilize signal-to-noise-ratio metrics generated according to the disclosed methods to generate and/or calibrate a quality reference table correlating a distribution of quality predictor values with a plurality of quality metrics. By improving the determination of signal-to-noise ratio metrics, the disclosed systems can improve the accuracy of these base-calling applications.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description refers to the drawings briefly described below.

FIG. 1 illustrates an environment in which an intensity correction and quality calibration system can operate in accordance with one or more embodiments of the present disclosure.

FIG. 2 illustrates the intensity correction and quality calibration system generating and utilizing a signal-to-noise-ratio metric in accordance with one or more embodiments of the present disclosure.

FIG. 3 further illustrates the intensity correction and quality calibration system generating a signal-to-noise-ratio metric in accordance with one or more embodiments of the present disclosure.

FIG. 4 further illustrates the intensity correction and quality calibration system utilizing one or more intensity correction parameters to generate a signal-to-noise-ratio metric in accordance with one or more embodiments of the present disclosure.

FIG. 5 illustrates the intensity correction and quality calibration system generating a quality metric for a nucleobase call in accordance with one or more embodiments of the present disclosure.

FIG. 6 illustrates the intensity correction and quality calibration system generating or calibrating a quality reference table in accordance with one or more embodiments of the present disclosure.

FIG. 7 illustrates comparative experimental results of generating quality metrics for two sequencing runs utilizing (i) an existing sequencing system and (ii) the intensity correction and quality calibration system in accordance with one or more embodiments of the present disclosure.

FIGS. 8A-8B illustrate comparative experimental results of generating signal-to-noise-ratio metric across a series of sequencing cycles utilizing (i) an existing sequencing system and (ii) the intensity correction and quality calibration system in accordance with one or more embodiments of the present disclosure.

FIGS. 9A-9B illustrate comparative experimental results of generating quality metrics across a series of sequencing cycles utilizing (i) an existing sequencing system and (ii) the intensity correction and quality calibration system in accordance with one or more embodiments of the present disclosure.

FIG. 10 illustrates a flowchart of a series of acts for generating a signal-to-noise-ratio metric for a sequencing cycle and generating, based on the generated signal-to-noise-ratio metric, a quality metric for a nucleobase call in accordance with one or more embodiments of the present disclosure.

FIG. 11 illustrates a block diagram of an example computing device for implementing one or more embodiments of the present disclosure.

### DETAILED DESCRIPTION

This disclosure describes embodiments of an intensity correction and quality calibration system that can determine an improved signal-to-noise-ratio metric for light signals emitted from fluorescent tags of nucleotide bases during a sequencing run and use such signal-to-noise-ratio metrics to determine more accurate nucleobase calls and base call quality. In particular, the intensity correction and quality calibration system can determine individual signal-to-noise-ratio metrics for various clusters of oligonucleotides to which tagged nucleotide bases are added. For instance, the intensity correction and quality calibration system can utilize the intensity values associated with the light signal emitted from a cluster to determine its corresponding signal-to-noise-ratio metric. In particular, the intensity correction and quality calibration system can determine per-cluster signal-to-noise-ratio metrics across successive sequencing cycles utilizing a direct approach to estimate both signal and noise components of the signal-to-noise-ratio metric for each respective sequencing cycle.

In some embodiments, for example, the intensity correction and quality calibration system determines each component of the signal-to-noise-ratio metric based directly on maximum-likelihood model estimates (e.g., of intensity correction parameters utilized to adjust intensity values of sequencing signals) for each respective sequencing cycle. In one or more embodiments, the intensity correction and quality calibration system derives noise levels for determining signal-to-noise-ratio metrics based on a mean squared sum of channel-specific error estimates generated based on intensity values for a signal corresponding to a target sequencing cycle utilizing a maximum likelihood estimation model. The intensity correction and quality calibration system can further determine a per-cluster signal-to-noise-ratio metric based on a scaling factor (e.g., a weighted amplification coefficient also generated utilizing the maximum likelihood estimation model) and the estimated noise level for the target sequencing cycle.

Moreover, the intensity correction and quality calibration system can utilize such improved signal-to-noise-ratio metrics associated with the clusters for a variety of base-calling applications. For example, the intensity correction and quality calibration system can use such signal-to-noise-ratio metrics to generate intensity-value boundaries for differentiating signals corresponding to different nucleotide bases according to a base-call-distribution model (e.g., segmented Gaussian mixture model), filter out clusters of poor quality, and/or determine a quality metric for nucleobase calls.

Also, the intensity correction and quality calibration system can utilize signal-to-noise-ratio metrics generated according to the disclosed methods to generate and/or calibrate a quality reference table correlating a distribution of quality predictor values with a plurality of quality metrics. Such a quality reference table, for example, can include a statistical representation of quality metrics derived from various well-characterized genomic samples sequenced according to the disclosed methods. Thus, the intensity correction and quality calibration system can use signal-to-noise-ratio metrics to provide a reference table for subsequent sequencing applications. Alternatively or additionally, the intensity correction and quality calibration system can utilize signal-to-noise-ratio metrics generated by the disclosed methods for such well-characterized samples to train a base-call-quality model for use in subsequent sequencing applications.

As mentioned, the intensity correction and quality calibration system provides various advantages over existing sequencing systems. For instance, the intensity correction and quality calibration system implements a new, unconventional approach to determining per-cluster signal-to-noise-ratio metrics across successive sequencing cycles of a sequencing run. Indeed, where existing, state-of-the-art systems implement an indirect approach to determine similar metrics for a target sequencing cycle based at least in part on a rolling average of noise levels (e.g., corrected intensity values) from previous cycles in the respective sequencing run, the intensity correction and quality calibration system implements an unconventional approach for determining signal-to-noise-ratio metrics based directly on intensity correction parameters generated for each respective cycle utilizing a maximum likelihood estimation model.

To illustrate, the intensity correction and quality calibration system implements an unconventional ordered combination of steps that involves direct derivation of noise levels for determining signal-to-noise-ratio metrics based on a mean squared sum of channel-specific error estimates generated based on intensity values for a signal corresponding to a target sequencing cycle utilizing a maximum likelihood estimation model. The intensity correction and quality calibration system can further determine a per-cluster signal-to-noise-ratio metric based on a scaling factor (e.g., a weighted amplification coefficient also generated utilizing the maximum likelihood estimation model) and the estimated noise level for the target sequencing cycle.

By utilizing this unconventional, direct approach, the intensity correction and quality calibration system can determine more accurate signal-to-noise-ratio metrics for sequencing cycles when compared to existing systems. In particular, the intensity correction and quality calibration system provides for accelerated convergence of the signal-to-noise-ratio metrics in early sequencing cycles of a sequencing run. Thus, the early signal-to-noise-ratio metrics determined by the intensity correction and quality calibration system are more reliable when compared to corresponding metrics determined by many existing systems. Further, by implementing this direct approach, the intensity correction and quality calibration system avoids the potentially detrimental influences from earlier cycles when determining the signal-to-noise-ratio metrics—a problem that often plagues many existing systems.

The intensity correction and quality calibration system can implement this new direct approach without any additional memory costs. For example, incorporation of the mean squared sum of channel-specific error estimates inherently requires the use of memory resources, potentially leading to significant additional memory consumption when compared to existing systems. The intensity correction and quality calibration system, however, implements this new approach without adding to the memory burden of existing systems. For instance, in some cases, the intensity correction and quality calibration system configures (e.g., compresses the bits representing) the mean squared sum of channel-specific error estimates to occupy the same space within memory as those values obtained by existing systems outside the channel estimations. Thus, the intensity correction and quality calibration system can directly swap the values used by existing systems with the mean squared sum of channel-specific error estimates without requiring additional memory.

Furthermore, by utilizing the improved signal-to-noise-ratio metric, the intensity correction and quality calibration system improves nucleobase calling. For example, as discussed above, the intensity correction and quality calibration system fits the base-call-distribution models used for generating nucleobase calls to various signal-to-noise-ratio ranges. These base-call-distribution models provide intensity-value boundaries (e.g., decision boundaries) upon which nucleotide-base calls are based. Thus, the intensity correction and quality calibration system flexibly tailors the intensity-value boundaries to the various levels of signal purity associated with the signals detected from sections of the nucleotide-sample slide. Accordingly, the intensity correction and quality calibration system can improve nucleobase calls for sections of the nucleotide-sample slide using intensity-value boundaries that are appropriate for their emitted signals, resulting in more accurate nucleotide-base calls.

By utilizing the improved signal-to-noise-ratio metric, the intensity correction and quality calibration system can also filter out poor-quality base calls for sections of a nucleotide-sample slide. In particular, the intensity correction and quality calibration system more accurately identifies sections of the nucleotide-sample slide that are emitting poor signals. Indeed, the intensity correction and quality calibration system can identify those sections of the nucleotide-sample slide that would otherwise pass a chastity filter implemented by conventional sequencing platforms only to surface their errors in later sequencing cycles. By improving the filtering process, the intensity correction and quality calibration system generates more accurate, more reliable nucleobase-call data.

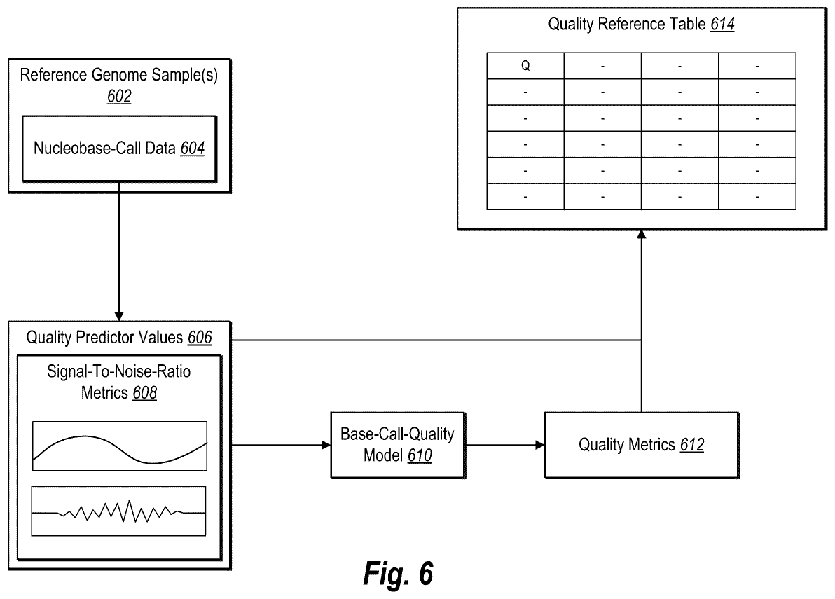

In addition to improved nucleobase calls and improved filtering, the intensity correction and quality calibration system more accurately determines nucleobase-call quality than existing sequencing systems. Indeed, by utilizing the improved signal-to-noise-ratio metric, the intensity correction and quality calibration system can more accurately estimate the quality of a nucleobase call. For example, as mentioned above, the intensity correction and quality calibration system can provide the improved signal-to-noise-ratio metric of a section of a nucleotide-sample slide as input to a base-call-quality model (e.g., a Phred model or a quality reference table). Moreover, as further described below (e.g., in relation to FIG. 6), the intensity correction and quality calibration system can utilize the improved signal-to-noise-ratio metric in conjunction with well-characterized genomic samples to generate and/or recalibrate a quality reference table associating quality metrics with various quality predictor values as a look-up reference for subsequence sequencing analyses. Accordingly, the intensity correction and quality calibration system utilizes a novel and improved (and sometimes additional) indicator of nucleobase-call quality when compared to conventional sequencing platforms, allowing for more accurate quality estimates.

Further, by using intensity-value boundaries that are tailored to the characteristics of detected light signals, the quality estimations tied to those intensity-value boundaries are also tailored to the characteristics of the light signals. Further still, by utilizing the improved signal-to-noise-ratio metric to generate and/or calibrate a quality reference table, as mentioned above, the intensity correction and quality calibration system can improve the accuracy of nucleobase-calls in subsequent sequencing runs that implement such quality reference tables to quickly determine quality metrics based on quality predictor values corresponding to signal of a particular sequencing run.

As suggested by the foregoing discussion, this disclosure utilizes a variety of terms to describe features and benefits of the intensity correction and quality calibration system. Additional detail is hereafter provided regarding the meaning of these terms as used in this disclosure. As used in this disclosure, for instance, “nucleotide-sample slide” (or “nucleotide-sample substrate”) refers to a plate or substrate, such as a flow cell, comprising oligonucleotides for sequencing nucleotide sequences from genomic samples or other sample nucleic-acid polymers. In particular, a nucleotide-sample slide can refer to a substrate containing fluidic channels through which reagents and buffers can travel as part of sequencing. For example, in one or more embodiments, a flow cell (e.g., a patterned flow cell or non-patterned flow cell) may comprise small fluidic channels and oligonucleotide samples that can be bound to adapter sequences on the substrate. In other implementations, a nucleotide-sample slide can be an open substrate with one or more regions for oligonucleotide samples to be analyzed and the oligonucleotide samples may be positioned using charged pads or other means. In yet another implementation, the nucleotide-sample slide can be a membrane having a nanopore through which one or more oligonucleotide samples may pass.

As used herein, a flow cell or other nucleotide-sample slide can (i) include a device having a lid extending over a reaction structure to form a flow channel therebetween that is in communication with a plurality of reaction sites of the reaction structure and (ii) include a detection device that is configured to detect designated reactions that occur at or proximate to the reaction sites. A flow cell or other nucleotide-sample slide may include a solid-state light detection or “imaging” device, such as a Charge-Coupled Device (CCD) or Complementary Metal-Oxide Semiconductor (CMOS) (light) detection device. As one specific example, a flow cell may be configured to fluidically and electrically couple to a cartridge (having an integrated pump), which may be configured to fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleobases having the same or different fluorescent labels. The nucleobases may bind to the reaction sites of the flow cell, such as to corresponding oligonucleotides at the reaction sites. The cartridge and/or bioassay system may then illuminate the reaction sites using an excitation light source (e.g., solid-state light sources, such as light-emitting diodes (LEDs)). The excitation light may provide emission signals (e.g., light of a wavelength or wavelengths that differ from the excitation light and, potentially, each other) that may be detected by the light sensors of the flow cell.

Relatedly, as used herein, the term “section of a nucleotide-sample slide” (or “nucleotide-sample slide section”) refers to an area that is part of a nucleotide-sample slide. In particular, a section of a nucleotide-sample slide can refer to a discrete portion of a nucleotide-sample slide that differs from other portions of the nucleotide-sample slide. For instance, a section of a nucleotide-sample slide can include a well (e.g., a nanowell) of a patterned flow cell or a discrete subsection of a non-pattered flow cell (e.g., a subsection corresponding to a cluster). In some cases, a section of a nucleotide-sample slide includes a tile or a sub-tile having clusters of the same or similar oligonucleotide growing in parallel. In some cases, a section of a nucleotide-sample slide includes an individual cluster of oligonucleotides on a nucleotide-sample slide.

In addition, as used herein, the term “cluster of oligonucleotides” (or simply “cluster”) refers to a localized group or collection of DNA or RNA molecules on a nucleotide-sample slide, such as a flow cell, or other solid surface. In particular, a cluster includes tens, hundreds, thousands, or more copies of a cloned or the same DNA or RNA segment. For example, in one or more embodiments, a cluster includes a grouping of oligonucleotides immobilized in a section of a flow cell or other nucleotide-sample slide. In some embodiments, clusters are evenly spaced or organized in a systematic structure within a patterned flow cell. By contrast, in some cases, clusters are randomly organized within a non-patterned flow cell. A cluster of oligonucleotides can be imaged utilizing one or more light signals. For instance, an oligonucleotide-cluster image may be captured by a camera during a sequencing cycle of light emitted by irradiated fluorescent tags incorporated into oligonucleotides from one or more clusters on a flow cell.

Additionally, as used herein, the term “labeled nucleotide base” refers to a nucleotide base having a fluorescent or light-based indicator of the classification of the nucleotide base. In particular, a labeled nucleotide base can refer to a nucleotide base that incorporates a fluorescent or light-based indicator to identify the type of base (e.g., adenine, cytosine, thymine, or guanine). For example, in one or more embodiments, a labeled nucleotide base includes a nucleotide base having a fluorescent tag that emits a signal that identifies the base type.

Further, as used herein the term “signal” refers to a signal emitted, reflected, or otherwise communicated from a labeled nucleotide base or a group of labeled nucleotide bases (e.g., labeled nucleotide bases added to a cluster of oligonucleotides). In particular, a signal can refer to a signal indicating the type of base. For example, a signal can include a light signal emitted or reflected from a fluorescent tag of a nucleotide base or fluorescent tags of multiple nucleotide bases incorporated into oligonucleotides. In some implementations, the intensity correction and quality calibration system triggers the signal through an external stimulus, such as a laser or other light source. In some cases, the intensity correction and quality calibration system triggers the signal through some internal stimuli. Further, in some embodiments, the intensity correction and quality calibration system observes the signal using a filter applied when capturing an image of the nucleotide-sample slide (e.g., section of the nucleotide-sample slide). As suggested above, in certain instances, a signal includes an aggregate of the signals provided by each labeled nucleotide base added to individual oligonucleotides in a cluster of oligonucleotides.

As used herein, the term “channel” refers to a range or filter of light, intensity, or color used to detect and/or measure a signal from a cluster of oligonucleotides. For example, a channel can include a particular range of light, intensity, or color of a laser used to illicit a fluorescent signal from fluorescent tags on nucleobases incorporated into oligonucleotides within a cluster. In some embodiments, the intensity correction and quality calibration system utilizes a two-channel implementation by, for instance, using two different ranges of light, intensities, or colors to illicit signals from clusters per sequencing cycle and capturing two corresponding images of a region of a nucleotide-sample slide per sequencing cycle. The first and second images can capture the intensity values of the emitted signal from the clusters that correspond to first and second light ranges. In some embodiments, the intensity correction and quality calibration system can utilize a single channel implementation, three-channel implementation, or four-channel implementation.

As used herein, the term “intensity value” refers to a value indicating a characteristic or attribute of a signal emitted, reflected, or otherwise communicated from a labeled nucleobase or a group of labeled nucleobases from a cluster of oligonucleotides. In particular, an intensity value can refer to a value associated with a color intensity (e.g., wavelength) or a light intensity (e.g., brightness). In some cases, the intensity correction and quality calibration system captures several images of a cluster of oligonucleotides with labeled nucleobases using different channels. Thus, an intensity value of a signal can correspond to the intensity of the signal as observed through a particular channel. In one or more embodiments, the intensity value is a measured degree of intensity for a cluster of oligonucleotides at the predicted location, and the location-error-prediction system can accordingly be applied to 16 quadrature amplitude modulation (QAM) modulation or pulse amplitude modulation (PAM) 4 modulation (e.g., using amplitude to encode base-call information).

Additionally, as used herein, the term “expected intensity value” refers to a value indicating an illumination state of a signal associated with a cluster of oligonucleotides (e.g., ON/OFF). In particular, an expected intensity value includes an expected value indicating an illumination state (e.g., ON/OFF) by a particular nucleobase (A, C, G, T) in a particular channel. For instance, in some cases, the expected intensity value refers to an average of (or centroid for) intensity values associated with the ON/OFF status of a particular channel. In certain implementations, the expected intensity value is an average of intensity values falling within the intensity-value boundaries (e.g., nucleotide clouds) of a certain base (A, C, G, or T). In certain implementations, the centroid value of the intensity channels is based on the intensity values of one or more sequencing cycles. In some embodiments, the expected intensity value is the same for all clusters of oligonucleotides within the region.

Additionally, as used herein, the term “intensity-value boundaries” refers to decision boundaries used in generating a nucleotide-base call for a signal. In particular, intensity-value boundaries can refer to decision boundaries that classify a nucleotide base (e.g., as A, T, C, or G) based on one or more intensity values of the signal. To illustrate, intensity-value boundaries can define or otherwise indicate the boundaries of a nucleotide cloud corresponding to each of the nucleotide bases. In some implementations, intensity-value boundaries do not mark the limits at which a signal is classified as a nucleotide base, but rather a point at which the signal can be classified as the nucleotide base with a particular level of accuracy.

As used herein, the term “intensity correction parameter” refers to adjustments made to correct or normalize the intensity values in data, such as the intensity values related to a light signal of a given sequencing cycle. For example, intensity correction parameters can include one or more of the following: distribution intensities defining intensity values at a centroid of base-specific intensity distributions, intensity errors representing differences between measured intensity values and corresponding distribution intensities, distribution centroid-to-origin distances, or distribution error-to-error similarity measures between distribution intensities and respective intensity errors. Relatedly, as used herein, the term “intensity error estimation” refers to an intensity correction parameter that approximates the errors or uncertainties associated with intensity values in data, such as the quantity of error associated with intensity values related to a given sequencing signal.

Also, as used herein, the term “variation correction coefficient” refers to specific values applied to measured intensity values to determine corrected intensity values. To illustrate, measured intensity values can be modeled as a function of a distribution intensity (e.g., of a particular intensity channel) for a given sequencing cycle and variation correction coefficients including a scaling factor, one or more correction offset factors, and/or the additive noise at the given sequencing cycle. Relatedly, as used herein, the term “scaling factor” (also referred to as an “amplification coefficient”) refers to a coefficient or value that indicates brightness. In particular, as used herein, the term scaling factor can refer to a value that accounts for scale variation (e.g., amplitude/brightness variation) in an inter-cluster intensity profile variation (which relates to the difference in scale and shifts from an origin of a multi-dimensional space of the intensity profiles of clusters in a cluster population). Also, as used herein, the term “correction offset factor” refers to a coefficient or value that indicates a lateral shift between a measured intensity and a corrected intensity value.

As used herein, the term “least squares solution” refers to a method utilized to optimize and find a best-fitting solution to a set of data points or functions. In particular, a least squares solution can be used to minimize the sum of the squares of differences between the observed values (e.g., observed intensity values for a given signal) and corresponding predicted or expected values (e.g., corrected intensity values). Relatedly, as used herein, the term “maximum likelihood estimation model” refers to a model used to estimate the parameters of a statistical model such as, for example, a least squares solution for intensity correction parameters and variation correction coefficients corresponding to a sequencing signal.

Additionally, as used herein, the term “signal-to-noise-ratio metric” refers to a measure of a target signal compared to a level or content of noise. In particular, a signal-to-noise-ratio metric can refer to the strength of a light signal that is detected from labeled nucleotide bases compared to associated noise. For example, in some implementations, a signal-to-noise-ratio metric includes a ratio of a scaling factor associated with a signal compared to the corresponding noise level. In one or more embodiments, the intensity correction and quality calibration system equates the scaling factor determined for a light signal to the light signal itself (e.g., the signal purity without the addition of noise). Further, as used herein, the term “noise level” refers to a value indicating the noise associated with a signal. In one or more embodiments, as will be discussed in more detail below, the intensity correction and quality calibration system determines the scaling factor and the noise level using one or more intensity values of the signal. As used herein, the term “signal-to-noise-ratio range” refers to a range of signal-to-noise-ratio metrics. In other words, in some implementations, the intensity correction and quality calibration system establishes one or more signal-to-noise-ratio ranges and determines whether the signal-to-noise-ratio metric of a signal falls within a particular signal-to-noise-ratio range.

Further, as used herein, the term “signal-to-noise-ratio threshold” refers to a threshold value established for filtering out a cluster of oligonucleotides (e.g., nucleotide-base calls associated with the cluster of oligonucleotides) based on the signal-to-noise-ratio metric. For example, in some implementations, the intensity correction and quality calibration system determines a signal-to-noise-ratio threshold as a signal-to-noise-ratio value that must be satisfied (e.g., met or exceeded) by a signal from labeled nucleotide bases corresponding to a cluster of oligonucleotides to have nucleotide-base calls for the cluster to be included in the resulting nucleotide-base-call data.

As further used herein, the term “nucleobase call” (or “nucleotide-base call” or simply “base call”) refers to a determination or prediction of a particular nucleobase (or nucleobase pair) for an oligonucleotide (e.g., nucleotide read) during a sequencing cycle or for a genomic coordinate of a genomic sample. In particular, a nucleobase call can indicate a determination or prediction of the type of nucleobase that has been incorporated within an oligonucleotide on a nucleotide-sample slide (e.g., read-based nucleobase calls). In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). As suggested above, a single nucleobase call can be an adenine (A) call, a cytosine (C) call, a guanine (G) call, a thymine (T) call, or an uracil (U) call. Note that the terms “nucleobase” and “nucleotide base” are interchangeable.

As used herein, the term “sequencing run” refers to an iterative process on a sequencing device to determine a primary structure of nucleotide sequences from a sample (e.g., genomic sample). In particular, a sequencing run includes cycles of sequencing chemistry and imaging performed by a sequencing device (including an imaging device, such as a CCD or CMOS) that incorporate nucleobases into growing oligonucleotides to determine nucleotide reads from nucleotide sequences extracted from a sample (or other sequences within a library fragment) and seeded throughout a flow cell. In some cases, a sequencing run includes replicating oligonucleotides derived or extracted from one or more genomic samples seeded in clusters throughout a flow cell. Upon completing a sequencing run, a sequencing device can generate base-call data in a file, such as a binary base call (BCL) sequence file or a fast-all quality (FASTQ) file.

As used herein, the term “sequencing cycle” (or “cycle”) refers to an iteration of adding or incorporating one or more nucleobases to one or more oligonucleotides representing or corresponding to sample's sequence (e.g., a genomic or transcriptomic sequence from a sample) or a corresponding adapter sequence. In some cases, a sequencing cycle includes an iteration of both incorporating nucleobases into clusters of oligonucleotides using sequencing chemistry and capturing images of such clusters attached to a nucleotide-sample slide (e.g., a flow cell). Accordingly, cycles can be repeated as part of sequencing a nucleic-acid polymer (e.g., a sample genomic sequence). For example, in one or more embodiments, each sequencing cycle involves incorporating nucleobases into either a single nucleotide read in which DNA or RNA strands are read in only a single direction or paired-end reads in which DNA or RNA strands are read from both ends but in different cycles. Further, in certain cases, each sequencing cycle involves a camera taking an image of the nucleotide-sample slide or multiple sections of the nucleotide-sample slide to generate image data for determining a particular nucleobase added or incorporated into particular oligonucleotides. Following the image capture stage, a sequencing system can remove certain fluorescent labels from incorporated nucleobases and perform another sequencing cycle until the nucleic-acid polymer has been completely sequenced. In one or more embodiments, a sequencing cycle includes a cycle within an SBS run. A sequencing cycle can include one or both of an indexing cycle and a genomic sequencing cycle. For instance, one cluster of oligonucleotides or a set of clusters of oligonucleotides may be undergoing a genomic sequencing cycle in which nucleobases corresponding to a sample genomic sequence are incorporated and another cluster of oligonucleotides or another set of clusters of oligonucleotides may be concurrently undergoing an indexing cycle in which nucleobases corresponding to an indexing sequence for a nucleotide read are incorporated.

Additionally, as used herein, the term “nucleobase-call data” refers to a digital file, image data, or other digital information indicating individual nucleotide bases or the sequence of nucleotide bases for a nucleic-acid polymer. In particular, nucleobase-call data can include intensity values (e.g., color or light intensity values for individual clusters) from images taken by a camera of a nucleotide-sample slide or other data that indicate individual nucleotide bases or the sequence of nucleotide bases for a nucleic-acid polymer. In addition, or in the alternative to intensity values, the nucleobase-call data may include chromatogram peaks or electrical current changes indicating individual nucleobases in a sequence. Additionally, in some embodiments, nucleobase-call data includes individual nucleobase calls identifying the individual nucleotide bases (e.g., A, T, C, or G). For example, nucleobase-call data can comprise data for nucleobase calls in a sequence for a nucleic-acid polymer, the number of nucleobase calls corresponding to a particular base (e.g., adenine, cytosine, thymine, or guanine), as organized in a digital file, such as a Binary Base Call (BCL) file. Further, nucleobase call data can include error/accuracy information, such as a quality metric associated with each nucleotide-base call. In some embodiments, nucleobase-call data comprises information from a sequencing device that utilizes sequencing by synthesis (SBS).

As used herein, the term “quality metric” (sometimes referred to as a “quality score”) refers to a specific score or other measurement indicating the accuracy of a nucleotide-base call for a sequencing cycle. In particular, a quality metric comprises a value indicating (i) a confidence in a base call or (ii) a probability of an error in the base call. For instance, a quality metric can include a numerical value where a relatively higher value indicates a relatively higher confidence in the accuracy of the corresponding base call. To illustrate, in some embodiments, a quality score includes a PHil's Read EDitor (PHRED) quality score (also referred to as a Q score) predicting the error probability of a given nucleotide-base call within a sequencing cycle.

As used herein, the term “quality reference table” refers to a data structure or model listing combinations of quality predictor values in relation to corresponding quality metrics based on empirical data related to well-characterized genomic samples. In particular, a quality reference table can include a data structure or model that maps combinations of quality predictor values to corresponding quality metrics. For instance, in some cases, the intensity correction and quality calibration system uses a quality reference table as a look-up table to identify a quality metric for a base call based on one or more quality predictor values associated with the base call. Relatedly, as used herein, the term “quality predictor value” refers to observable properties of sequencing data related to a nucleobase call, such as but not limited to intensity values, signal-to-noise-ratio metrics, chastity values, and other characteristics related to base call quality. Also, as used herein, the term “chastity value” (sometimes referred to as a “chastity metric”) refers to a measure of reliability of signals detected from a cluster of oligonucleotides. For instance, in some cases, a chastity value refers to the ratio of the brightest base intensity of a signal and the sum of the brightest and second brightest base intensities thereof. In some instances, the intensity correction and quality calibration system uses chastity values as a filter to remove unreliable clusters from the analysis results. To illustrate, in some cases, the intensity correction and quality calibration system removes a cluster from the analysis results if a predetermined number of base calls from the cluster have a chastity value below some established threshold. The intensity correction and quality calibration system can use other definitions for a chastity value in various embodiments. In some cases, for example, a chastity value can be determined for a corresponding signal as the ratio of a distance between the intensity associated with the signal and the nearest nucleobase centroid to ta distance between the intensity and another centroid (e.g., the second nearest centroid).

As used herein, the term “base-call-quality model” refers to a computer model or algorithm that generates a quality metric for a nucleotide-base call. For example, a base-call-quality model can refer to a computer algorithm that analyzes characteristics of a signal and/or the corresponding cluster or labeled nucleotide bases and generates a quality metric for the nucleotide-base call based on the analysis. To illustrate, in some implementations, the base-call-quality model includes a computer algorithm that generates a Phred quality score. Alternatively, in some implementations, the base-call-quality model comprises a quality reference table which correlates one or more quality predictor values, including signal-to-noise-ratio metrics, with quality metrics, as further defined and described below. In some instances, a base-call-quality model includes a model or algorithm (e.g., a Phred algorithm) used for generating or calibrating such a quality reference table.

As used herein, the term “base-call-distribution model” refers to a computer model or algorithm that generates intensity-value boundaries. For example, in some implementations, a base-call-distribution model includes, but is not limited to, a Gaussian distribution model, a uniform distribution model, a Bernoulli distribution model, a binomial distribution model, or a Poisson distribution model. As used herein, the term “centroid” refers to the center of a nucleotide cloud defined or otherwise indicated by one or more intensity-value boundaries. Further, as used herein, the term “centroid intensity value” refers to an intensity value associated with a centroid. In particular, a centroid intensity value indicates an intensity value that corresponds to the center of a nucleotide cloud.

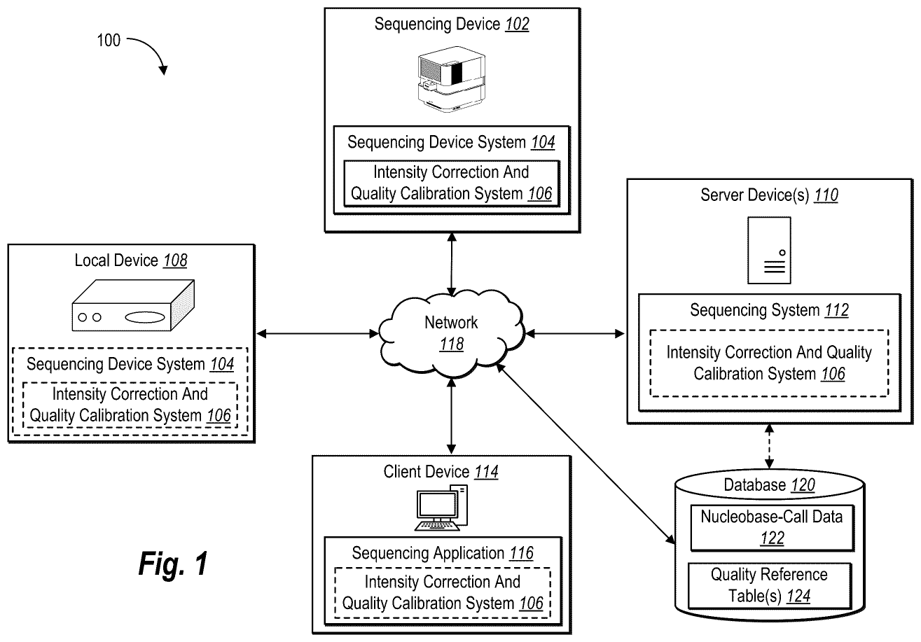

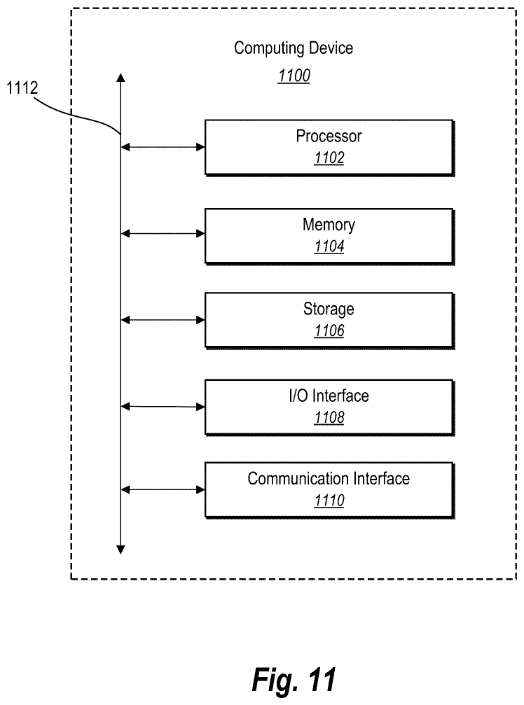

The following paragraphs describe the intensity correction and quality calibration system with respect to illustrative figures that portray example embodiments and implementations. For example, FIG. 1 illustrates a schematic diagram of a computing system 100 in which an intensity correction and quality calibration system 106 operates in accordance with one or more embodiments. As illustrated, the computing system 100 includes a sequencing device 102 connected to a local device 108 (e.g., a local server device), one or more server device(s) 110, a client device 114, and a database 120. As shown in FIG. 1, the sequencing device 102, the local device 108, the server device(s) 110, the client device 114, and the database 120 can communicate with each other via a network 118. The network 118 comprises any suitable network over which computing devices can communicate. Example networks are discussed in additional detail below with respect to FIG. 11. While FIG. 1 shows an embodiment of the intensity correction and quality calibration system 106, this disclosure describes alternative embodiments and configurations below.

As indicated by FIG. 1, the sequencing device 102 comprises a computing device and a sequencing device system 104 for sequencing a genomic sample or other nucleic-acid polymer. In some embodiments, by executing the sequencing device system 104 using a processor, the sequencing device 102 analyzes nucleotide fragments or oligonucleotides extracted from genomic samples to generate nucleotide reads or other data utilizing computer implemented methods and systems either directly or indirectly on the sequencing device 102. More particularly, the sequencing device 102 receives nucleotide-sample slides (e.g., flow cells) comprising nucleotide fragments extracted from samples and further copies and determines the nucleobase sequence of such extracted nucleotide fragments.

In one or more embodiments, the sequencing device 102 utilizes sequencing-by-synthesis (SBS) techniques to sequence nucleotide fragments into nucleotide reads and determine nucleobase calls for the nucleotide reads. In addition or in the alternative to communicating across the network 118, in some embodiments, the sequencing device 102 bypasses the network 118 and communicates directly with the local device 108, the client device 114, and/or the database 120. By executing the sequencing device system 104, the sequencing device 102 can further store the nucleobase calls as part of base-call data that is formatted as a binary base call (BCL) file and send the BCL file to the local device 108, the server device(s) 110, and/or the database 120.

As further indicated by FIG. 1, the local device 108 is located at or near a same physical location of the sequencing device 102. Indeed, in some embodiments, the local device 108 and the sequencing device 102 are integrated into a same computing device. The local device 108 may run the sequencing device system 104 and/or the intensity correction and quality calibration system 106 to generate, receive, analyze, store, and transmit digital data, such as by receiving base-call data or determining variant calls based on analyzing such base-call data. As shown in FIG. 1, the sequencing device 102 may send (and the local device 108 may receive) base-call data generated during a sequencing run of the sequencing device 102. The local device 108 may also communicate with the client device 114. In particular, the local device 108 can send data to the client device 114, including a binary alignment map (BAM) file, a variant call format (VCF) file, or other information indicating nucleobase calls, sequencing metrics, error data, or other metrics.

As further indicated by FIG. 1, the server device(s) 110 is located remotely from the local device 108 and the sequencing device 102. Additionally, the server device(s) 110 include a sequencing system 112 for receiving, generating, storing, and/or processing sequencing data. Further, similar to the local device 108, in some embodiments, the server device(s) 110 include a version of (or are otherwise able to access or implement) the intensity correction and quality calibration system 106. Accordingly, the server device(s) 110 may generate, receive, analyze, store, and transmit digital data, such as by receiving base-call data or determining variant calls based on analyzing such base-call data. As indicated above, the sequencing device 102 may send (and the server device(s) 110 may receive) base-call data from the sequencing device 102. The server device(s) 110 may also communicate with the client device 114. In particular, the server device(s) 110 can send data to the client device 114, including BAM files, VCF files, or other sequencing related information.

In some embodiments, the server device(s) 110 comprise a distributed collection of servers where the server device(s) 110 include a number of server devices distributed across the network 118 and located in the same or different physical locations. Further, the server device(s) 110 can comprise a content server, an application server, a communication server, a web-hosting server, or another type of server. Moreover, as shown in FIG. 1, the server device(s) 110 are in communication, either directly or via the network 118, with the database 120 storing, among other things, nucleobase-call data 122 associated with one or more sequencing runs and one or more quality reference tables 124 generated and/or calibrated by the intensity correction and quality calibration system 106 (e.g., as described in relation to FIG. 6).

As indicated above, as part of the server device(s) 110 or the local device 108, the intensity correction and quality calibration system 106 can generate, encode, and/or utilize the nucleobase-call data 122 and/or the one or more quality reference tables 124. In some embodiments, for example, the intensity correction and quality calibration system 106 can utilize the nucleobase-call data for a given sequencing cycle to determine an improved signal-to-noise-ratio metric and implement the signal-to-noise-ratio metric in downstream applications, as described in greater detail below in relation to the subsequent figures.

As further illustrated and indicated in FIG. 1, by executing a sequencing application 116, the client device 114 can generate, store, receive, and send digital data. In particular, the client device 114 can receive sequencing data from the local device 108 or receive call files (e.g., BCL) and sequencing metrics from the sequencing device 102. Furthermore, the client device 114 may communicate with the local device 108 or the server device(s) 110 to receive a VCF comprising genotype or variant calls and/or other metrics, such as base-call-quality metrics or pass-filter metrics. The client device 114 can accordingly present or display information pertaining to variant calls or other genotype calls within a graphical user interface of the sequencing application 116 to a user associated with the client device 114. For example, the client device 114 can present nucleobase calls, genotype calls, variant calls, and/or sequencing metrics for a sequenced genomic sample within a graphical user interface of the sequencing application 116.

Although FIG. 1 depicts the client device 114 as a desktop or laptop computer, the client device 114 may comprise various types of client devices. For example, in some embodiments, the client device 114 includes non-mobile devices, such as desktop computers or servers, or other types of client devices. In yet other embodiments, the client device 114 includes mobile devices, such as laptops, tablets, mobile telephones, or smartphones. Additional details regarding the client device 114 are discussed below with respect to FIG. 11.

As further illustrated in FIG. 1, the client device 114 includes the sequencing application 116. The sequencing application 116 may be a web application or a native application stored and executed on the client device 114 (e.g., a mobile application, desktop application). The sequencing application 116 can include instructions that (when executed) cause the client device 114 to receive data from the intensity correction and quality calibration system 106 and present, for display at the client device 114, base-call data or data from an alignment data file or VCF. Furthermore, the sequencing application 116 can instruct the client device 114 to display summaries for multiple sequencing runs.

As further illustrated in FIG. 1, a version of the intensity correction and quality calibration system 106 may be located and/or implemented (e.g., entirely or in part) on the client device 114 or the sequencing device 102. In yet other embodiments, the intensity correction and quality calibration system 106 is implemented by one or more other components of the computing system 100, such as the local device 108. In particular, the intensity correction and quality calibration system 106 can be implemented in a variety of different ways across the sequencing device 102, the local device 108, the server device(s) 110, and the client device 114. For example, the intensity correction and quality calibration system 106 can be downloaded from the server device(s) 110 to the client device 114 and/or the local device 108 where all or part of the functionality of the intensity correction and quality calibration system 106 is performed at each respective device within the computing system 100.

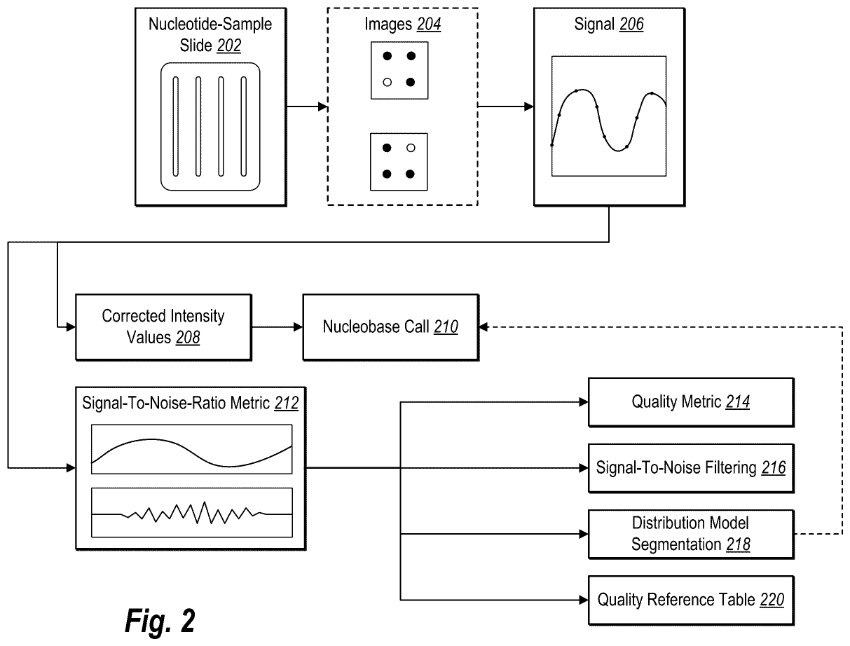

As mentioned previously, in some embodiments, the intensity correction and quality calibration system 106 generates improved signal-to-noise-ratio metrics for a series of signals detected from a section of a nucleotide-sample slide. In particular, the intensity correction and quality calibration system 106 generates a signal-to-noise-ratio metric for a respective signal from a given sequencing cycle of a series of sequencing cycles based on intensity correction parameters derived from intensity values for the respective signal. The intensity correction and quality calibration system 106 can utilize the signal-to-noise-ratio metric to provide various nucleobase-calling and related downstream features. To illustrate, FIG. 2 depicts an overview diagram of the intensity correction and quality calibration system 106 generating and utilizing a signal-to-noise-ratio metric in accordance with one or more embodiments.

As shown in FIG. 2, the intensity correction and quality calibration system 106 utilizes a nucleotide-sample slide 202 for sequencing of nucleotide bases. As described above, the nucleotide-sample slide 202 can include oligonucleotides that receive or incorporate labeled nucleotide bases. In particular, the nucleotide-sample slide 202 can include a cluster of oligonucleotides with each section (e.g., well) thereof. When stimulated, the labeled nucleotide bases can emit a signal having characteristics associated with the type of nucleotide base.

As further shown in FIG. 2, the intensity correction and quality calibration system 106 captures a series of images 204 of at least one section of the nucleotide-sample slide 202, such as a section corresponding to an individual cluster of oligonucleotides. In particular, the intensity correction and quality calibration system 106 captures the series of image 204 as the labeled nucleotide bases within the section of the nucleotide-sample slide 202 emit a respective series of signals. As shown, in one or more embodiments, the intensity correction and quality calibration system 106 captures multiple images for each sequencing cycle in a series of sequencing cycles. For example, the intensity correction and quality calibration system 106 can utilize various image filters to capture the multiple images. For example, in some embodiments, the intensity correction and quality calibration system 106 utilizes a two-channel implementation, capturing two images of the section of the nucleotide-sample slide 202. In particular, the intensity correction and quality calibration system 106 captures a first image for a first channel using a first image filter and captures a second image for a second channel using a second image filter. Accordingly, the first and second images can capture an intensity of the emitted signal that corresponds to the image filter used. In some cases, the intensity correction and quality calibration system 106 utilizes a four-channel implementation to capture four different images of the section of the nucleotide-sample slide 202 for each sequencing cycle. Similar to the two-channel implementation, the intensity correction and quality calibration system 106 can capture each image for the four-channel implementation using a different image filter to capture an intensity of the emitted signal for each respective image. Thus, in some cases, each of the multiple images of the images 204 depicts the respective emitted signal with a different intensity.

As further shown in FIG. 2, the images 204 portray a signal 206 emitted from the labeled nucleotide bases located within the section of the nucleotide-sample slide 202 during the respective sequencing cycle. As mentioned, the signal 206 can indicate the type of nucleotide base that was added to the cluster of oligonucleotides with the section of the nucleotide-sample slide 202 for the given sequence cycle of a series of sequencing cycles. For example, as described in additional detail below, the signal 206 can have one or more corresponding intensity values that indicate the corresponding type of nucleotide base. In some implementations, for example, each of the images 204 captures at least one intensity value corresponding to the signal 206 for the given sequencing cycle of a series of sequencing cycles.

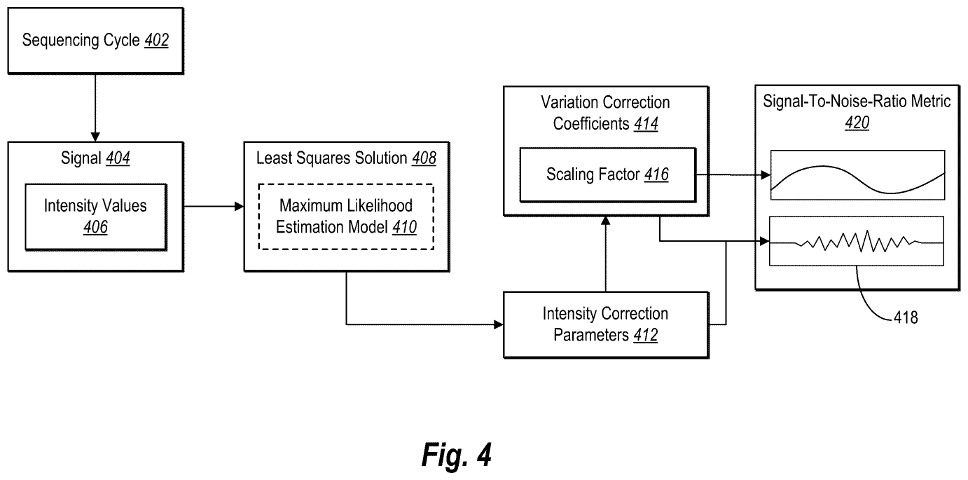

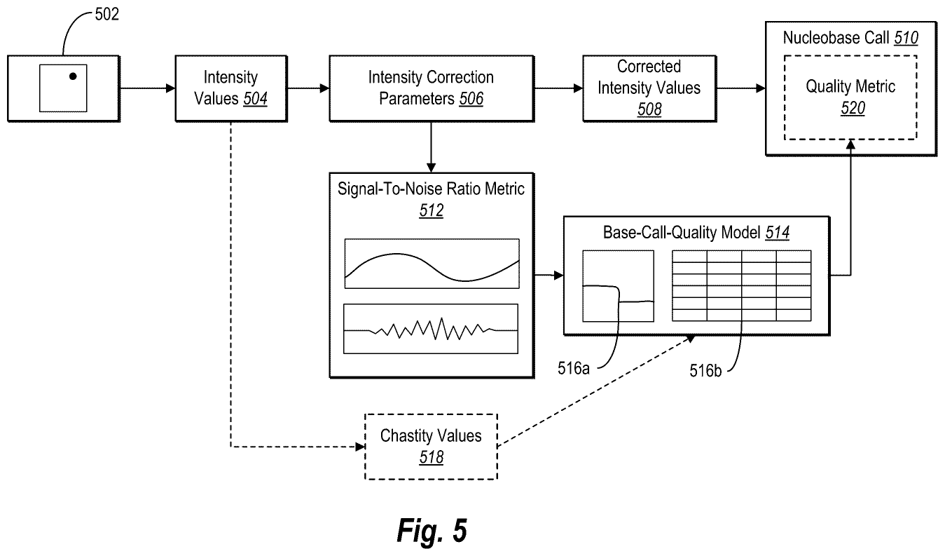

Moreover, in many implementations, the signal 206 comprises at least some associated noise. In particular, the signal 206 can have an associated noise level that affects the purity of the signal 206. Accordingly, as shown in FIG. 2, the intensity correction and quality calibration system 106 can generate corrected intensity values 208 for the signal 206 and determine a nucleobase call 210 for the signal 206 based on the corrected intensity values 208 (e.g., as further described below in relation to FIGS. 4-5). In some embodiments, for example, the intensity correction and quality calibration system 106 determines the corrected intensity values 208 for the signal 206 utilizing one or more of the methods described by Systems and Methods for Per-Cluster Intensity Correction and Base Calling, U.S. application Ser. No. 17/510,285 (filed Oct. 25, 2021), and Inter-Cluster Intensity Variation Correction and Base Calling, U.S. application Ser. No. 18/154,603 (filed Jan. 13, 2023), which are hereby incorporated by reference in their entirety.

To further illustrate, in one or more embodiments, the term “corrected intensity value” refers to an intensity value corresponding to a signal emitted from a section of a nucleotide-sample slide that has been adjusted based on one or more features of the signal. In one or more embodiments, for instance, a corrected intensity value includes an intensity value that has been corrected to account for offset and a scaling factor corresponding to an intensity value. Upon correction, in some cases, the corrected intensity value is closer to a centroid of a nucleotide cloud than the corresponding intensity value that was initially measured for the signal. For example, in a two-channel implementation, the intensity correction and quality calibration system 106 can determine a pair of corrected intensity values (e.g., one for each intensity channel) so that the pair is nearer to the centroid of a nucleotide cloud than the corresponding pair of intensity values initially measured for the signal.

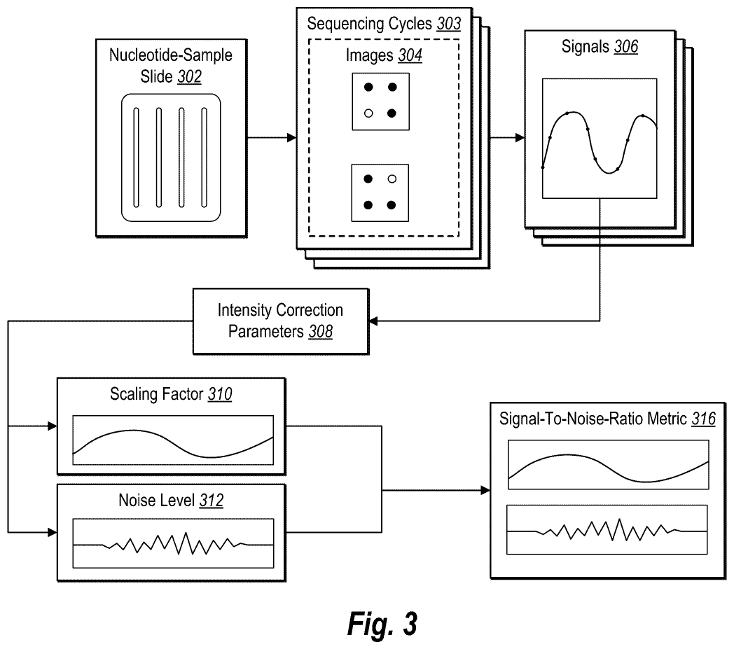

Furthermore, as also shown in FIG. 2, the intensity correction and quality calibration system 106 generates a signal-to-noise-ratio metric 212 for the signal 206. For instance, the intensity correction and quality calibration system 106 can generate a scaling factor corresponding to the signal 206. In one or more embodiments, the intensity correction and quality calibration system 106 equates the square of the determined scaling factor to the numerator of the signal-to-noise-ratio metric 212 (e.g., the “signal” of the signal-to-noise ratio). In addition, the intensity correction and quality calibration system 106 can determine a noise level corresponding to the signal 206 (e.g., as further described below in relation to FIGS. 3-4) and generate the signal-to-noise-ratio metric 212 utilizing the scaling factor and the noise level.

As also mentioned, the intensity correction and quality calibration system 106 can utilize the signal-to-noise-ratio metric 212 for various downstream base-calling features. As shown in FIG. 2, for example, the intensity correction and quality calibration system 106 can utilize the signal-to-noise-ratio metric 212 to determine a quality metric 214 of the nucleobase call 210 generated from the signal 206. For example, the intensity correction and quality calibration system 106 can utilize a base-call-quality model to determine the quality metric 214 based on the signal-to-noise-ratio metric 212.

As further shown in FIG. 2, the intensity correction and quality calibration system 106 can utilize the signal-to-noise-ratio metric 212 for signal-to-noise filtering 216. In particular, the intensity correction and quality calibration system 106 can establish a signal-to-noise-ratio threshold and exclude the signal 206 (e.g., the corresponding sequencing cycle or the corresponding section of the nucleotide-sample slide 202) from nucleobase-call data if the signal-to-noise-ratio metric 212 fails to satisfy the signal-to-noise-ratio threshold.

Additionally or alternatively, as shown in FIG. 2, the intensity correction and quality calibration system 106 can utilize the signal-to-noise-ratio metric 212 for distribution model segmentation 218. In particular, the intensity correction and quality calibration system 106 can utilize the signal-to-noise-ratio metric 212 to segment a base-call-distribution model—such as a Gaussian mixture model—into separate base-call-distribution models. In some implementations, the intensity correction and quality calibration system 106 segments the base-call-distribution model by fitting a separate base-call-distribution model to each of a plurality of signal-to-noise-ratio ranges. Indeed, the intensity correction and quality calibration system 106 can determine signal-to-noise-ratio metrics (including the signal-to-noise-ratio metric 212) for multiple series of signals detected from multiple sections of the nucleotide-sample slide 202. The intensity correction and quality calibration system 106 further determines a plurality of signal-to-noise-ratio ranges for the signal-to-noise-ratio metrics. Accordingly, the intensity correction and quality calibration system 106 can fit a base-call distribution to each of the signal-to-noise-ratio ranges.

Moreover, the intensity correction and quality calibration system 106 can further utilize the base-call-distribution model for a particular signal-to-noise-ratio range to generate nucleobase calls for the signals having a signal-to-noise-ratio metric that falls within that range. Accordingly, the intensity correction and quality calibration system 106 can utilize the signal-to-noise-ratio metric 212 to generate the nucleobase call 210 for the signal 206 via the distribution model segmentation 218.

As further shown in FIG. 2, the intensity correction and quality calibration system 106 can also utilize one or more signal-to-noise-ratio metrics generated according to the foregoing methods to generate and/or calibrate a quality reference table 220 correlating a distribution of quality predictor values with a plurality of quality metrics. Furthermore, the intensity correction and quality calibration system 106 can utilize a pre-calibrated quality reference table to generate a quality metric for a nucleobase call determined for a give signal.

Though much of the above discussion (as well as the following discussion) focuses on determining a signal-to-noise-ratio metric for a given signal of a series of signals corresponding to a particular section of a nucleotide-sample slide, it should be understood that the intensity correction and quality calibration system 106 can determine a signal-to-noise-ratio metric for each of a plurality of sections of the nucleotide-sample slide in parallel. For instance, in one or more embodiments, the intensity correction and quality calibration system 106 detects a series of signals from each section of the nucleotide-sample slide (e.g., each well or each section corresponding to a cluster of oligonucleotides) and determines signal-to-noise-ratio metrics for each detected series of signals. Thus, the intensity correction and quality calibration system 106 can utilize the various signal-to-noise-ratio metrics for determining quality metrics for generated nucleobase calls, for signal-to-noise filtering, for determining nucleobase calls via segmented base-call-distribution models, and/or for generating or calibrating a quality reference table. Furthermore, in some embodiments, the intensity correction and quality calibration system 106 can utilize signal-to-noise-ratio metrics to perform some or all of the functions described by Signal-To-Noise-Ratio Metric for Determining Nucleotide-Base Calls and Base-Call Quality, U.S. application Ser. No. 17/805,138 (filed Jun. 2, 2022), which is hereby incorporated by reference in its entirety.

As previously mentioned, in some embodiments, the intensity correction and quality calibration system 106 generates a signal-to-noise-ratio metric based on a scaling factor and a noise level determined for a given signal of a series of signals corresponding to a series of sequencing cycles. For example, FIG. 3 illustrates an overview of the intensity correction and quality calibration system 106 determining a scaling factor 310 and a noise level 312 for a given signal of a series of signals 306 from a respective series of sequencing cycles 303 and generating a signal-to-noise-ratio metric 316 for the respective signal. In particular, as shown in FIG. 3, the intensity correction and quality calibration system 106 derives the scaling factor 310 and the noise level 312 from intensity correction parameters 308 generated to determine one or more corrected intensity values for the given signal of the series of signals 306 (e.g., as mentioned above in relation to FIG. 2).

Accordingly, as shown in FIG. 3, the intensity correction and quality calibration system 106 captures, for each sequencing cycle of the series of sequencing cycles 303, images 304 of at least one section of a nucleotide-sample slide 302, such as images of a cluster of oligonucleotides within a section of the nucleotide-sample slide 302. For instance, a camera for the sequencing device 102—and associated with the intensity correction and quality calibration system 106—captures the images 304 of tiles within the nucleotide-sample slide 302, where each tile includes multiple nanowells comprising clusters or multiple subsections comprising clusters. As further shown, the images 304 portray a respective signal of the series of signal 306 emitted from the at least one section of the nucleotide-sample slide 302 (e.g., from the labeled nucleotide bases within a well or subsection corresponding to a cluster of oligonucleotides).

As further shown in FIG. 3, the intensity correction and quality calibration system 106 determines the intensity correction parameters 308 corresponding to a given signal of the series of signals 306. In some embodiments, for example, the intensity correction and quality calibration system 106 determines the intensity correction parameters 308 utilizing a least squares solution configured to determine corrected intensities for sequencing signals, such as described below in relation to FIG. 4. In particular embodiments, the intensity correction and quality calibration system 106 utilizes the least squares solution to determine variation correction coefficients based on the intensity correction parameters 308 corresponding to the given signal of the series of signals 306. In one or more embodiments, such as where a two-channel implementation is used, the variation correction coefficients include the scaling factor 310 that accounts for scale variation in an inter-cluster intensity profile and two correction offset factors (also referred to as channel-specific offset coefficients or channel-specific offset factors) that account for shift variation along the first and second intensity channels in the inter-cluster intensity profile variation, respectively.

Accordingly, the intensity correction and quality calibration system 106 can utilize a least squares solution (or similar method) to determine the intensity correction parameters 308 and the aforementioned variation correction coefficients by determining a relationship between a measured intensity for the labeled nucleotide bases (e.g., a measured intensity corresponding to the respective signal) and the variation correction coefficients. The intensity correction and quality calibration system 106 can further determine an error function based on the relationship between the measured intensity and the variation correction coefficients. Thus, the intensity correction and quality calibration system 106 can determine the scaling factor 310 by generating a partial derivative of the error function with respect to the scaling factor. In particular, in some implementations, the intensity correction and quality calibration system 106 utilizes the aforementioned least squares solution to determine two partial derivatives of the error function: one with respect to the scaling factor 310 and another with respect to the channel-specific offset factors.

As further shown in FIG. 3, the intensity correction and quality calibration system 106 determines the noise level 312 corresponding to the given signal of the series of signals 306. In particular, the intensity correction and quality calibration system 106 utilizes the intensity correction parameters 308 to determine the noise level 312 (e.g., as described in further detail below in relation to FIG. 4). Indeed, as mentioned above, the intensity correction and quality calibration system 106 determines the noise level 312 for the given signal of the series of signals 306 from the respective series of sequencing cycles 303 without reliance upon or influence from information from previous sequencing cycles of the series of sequencing cycles 303. Instead, as shown in FIG. 3, the intensity correction and quality calibration system 106 determines the noise level 312 utilizing the intensity correction parameters 308 corresponding to the given signal of the series of signals 306.

As shown in FIG. 3, the intensity correction and quality calibration system 106 utilizes the scaling factor 310 and the noise level 312 for the given signal of the series of signals 306 to determine the respective signal-to-noise-ratio metric 316. For example, the intensity correction and quality calibration system 106 can determine the signal-to-noise-ratio metric 316 utilizing a ratio of the square of the scaling factor 310 to the noise level 312. Indeed, in one or more embodiments, the intensity correction and quality calibration system 106 equates the square of the scaling factor 310 to the respective “signal” (e.g., the numerator) of the signal-to-noise-ratio metric 316.

In one or more embodiments, the intensity correction and quality calibration system 106 accounts for phasing or pre-phasing when determining the signal-to-noise-ratio metric for a signal. As used herein, the term “phasing” refers to an effect or situation where sequencing on one molecule falls at least one base behind other molecules at a particular cycle. Conversely, as used herein, the term “pre-phasing” refers to an effect or situation where sequencing on one molecule jumps at least one base ahead of other molecules at a particular cycle. In one or more embodiments, to correct for the effects of phasing or pre-phasing, the intensity correction and quality calibration system 106 can detect a signal with an intensity value for base incorporation at each cycle and correct the intensity value by (i) subtracting an intensity value of an immediately previous cycle from an intensity value of a current cycle and (ii) subtracting an intensity value of an immediately subsequent cycle from the intensity value of the current cycle. Indeed, in one or more embodiments, the intensity correction and quality calibration system 106 corrects the effects of phasing or pre-phasing as described in U.S. Pat. No. 10,689,696, issued Jun. 23, 2020, and entitled “Methods and Systems for Analyzing Image Data,” which is incorporated herein by reference in its entirety.

As mentioned previously, in some embodiments, the intensity correction and quality calibration system 106 generates an improved signal-to-noise-ratio metric based on a plurality of intensity correction parameters of a least squares solution configured to provide corrected intensity values for sequencing cycles. For example, FIG. 4 illustrates the intensity correction and quality calibration system 106 utilizing a least squares solution 408 to determine a plurality of intensity correction parameters 412 and/or one or more variation correction coefficients 414 for a sequencing cycle 402 and generating a signal-to-noise-ratio metric 420 based on the plurality of intensity correction parameters 412 and the one or more variation correction coefficients 414 output by the least squares solution 408.

As shown in FIG. 4, the intensity correction and quality calibration system 106 identifies (or receives) a signal 404 corresponding to the sequencing cycle 402. As mentioned, in some embodiments, the signal 404 corresponds to one or more images generated for the sequencing cycle 402. As illustrated, the intensity correction and quality calibration system 106 determines (or receives) intensity values 406 corresponding to the signal 404 (e.g., as described above in relation to FIG. 2).

As also shown in FIG. 4, the intensity correction and quality calibration system 106 can determine the plurality of intensity correction parameters 412 and/or the one or more variation correction coefficients 414 using a least squares solution 408 based on the intensity values 406. As mentioned above, the intensity correction and quality calibration system 106 can utilize the least squares solution 408 to determine the plurality of intensity correction parameters 412 and/or the one or more variation correction coefficients 414 by determining a relationship between the intensity values 406 and the plurality of intensity correction parameters 412. In particular, as illustrated, the intensity correction and quality calibration system 106 determines the one or more variation correction coefficients 414 based on the plurality of intensity correction parameters 412.

In one or more embodiments, the intensity correction and quality calibration system 106 utilizes a maximum likelihood estimation model 410 approaching the least squares solution 408 to determine the plurality of intensity correction parameters 412 and the one or more variation correction coefficients 414. In some cases, for instance, the intensity correction and quality calibration system 106 can utilize a maximum likelihood estimation model approaching a least squares solution as described by U.S. application Ser. No. 17/510,285 and/or U.S. application Ser. No. 18/154,603.

In some embodiments, for example, the intensity correction and quality calibration system 106 utilizes the maximum likelihood estimation model 410 to determine the plurality of intensity correction parameters 412 (\hat{x}₁, \hat{x}₂, x̅x̅, ê₁, ê₂, and x̅e̅) and the one or more variation correction coefficients 414 (scaling factor â, channel-specific correction offset factors d₁ and d₂) approaching the least squares solution 408 for a two-channel implementation as follows:

$${\hat{x}}_{1} {=}^{△} \frac{1}{C} {\sum }_{c = 1}^{C} {x}_{c , 1}$$

$${\hat{x}}_{2} {=}^{△} \frac{1}{C} {\sum }_{c = 1}^{C} {x}_{c , 2}$$

$$\overline{xx} {=}^{△} \frac{1}{C} {\sum }_{c = 1}^{C} ( {x}_{c , 1}^{2} + {x}_{c , 2}^{2} )$$

$${\hat{e}}_{1} {=}^{△} \frac{1}{C} {\sum }_{c = 1}^{C} {x}_{c , 1}$$

$${\hat{e}}_{2} {=}^{△} \frac{1}{C} {\sum }_{c = 1}^{C} {x}_{c , 2}$$

$$\overline{xe} {=}^{△} \frac{1}{C} {\sum }_{c = 1}^{C} ( {x}_{c , 1} {e}_{c , 1} + {x}_{c , 2} {e}_{c , 2} )$$

$${k}_{a} ( C ) = \frac{{σ}_{n}^{2}}{C {σ}_{a}^{2}}$$

$${k}_{{d}_{1}} ( C ) = \frac{C {σ}_{{d}_{1}}^{2}}{C {σ}_{{d}_{1}}^{2} + {σ}_{n}^{2}}$$

$${k}_{{d}_{2}} ( C ) = \frac{C {σ}_{{d}_{2}}^{2}}{C {σ}_{{d}_{2}}^{2} + {σ}_{n}^{2}}$$

$$\hat{a} = 1 + \frac{\overline{xe} - {\hat{x}}_{1} {\hat{e}}_{1} {k}_{{d}_{1}} ( C ) - {\hat{x}}_{2} {\hat{e}}_{2} {k}_{{d}_{2}} ( C )}{\overline{xx} + {k}_{a} ( C ) - {\hat{x}}_{1}^{2} {k}_{{d}_{1}} ( C ) - {\hat{x}}_{2}^{2} {k}_{{d}_{1}} ( C )}$$

$${d}_{1} = ( {\hat{e}}_{1} + {\hat{x}}_{1} ( 1 - \hat{a} ) ) {k}_{{d}_{1}} ( C )$$

$${d}_{2} = ( {\hat{e}}_{2} + {\hat{x}}_{2} ( 1 - \hat{a} ) ) {k}_{{d}_{2}} ( C )$$

where a, d₁, d₂, and n_{c,i} have prior probability distributions represented by a˜N(1, σₙ²), d₁˜N(0, σ_{d}_{₁}²), d₂˜N(0, σ_{d}_{₂}²), and n_{c,i}˜N(0, σₙ²), respectively, and e_{c,i}=y_{c,i}-x_{c,i}. Accordingly, as C→∞ in the portrayed embodiment, the plurality of intensity correction parameters 412 and the one or more variation correction coefficients 414 approach the least squares solution 408.

As further shown in FIG. 4, the intensity correction and quality calibration system 106 utilizes the plurality of intensity correction parameters 412 and the one or more variation correction coefficients 414 to determine a signal-to-noise-ratio metric 420 for the signal 404 corresponding to the sequencing cycle 402. As mentioned previously, for instance, the intensity correction and quality calibration system 106 can equate a scaling factor 416, represented by â in the above equations, of the one or more variation correction coefficients 414 to the respective signal for the signal-to-noise-ratio metric 420. In addition to utilizing the scaling factor 416 as a signal component of the signal-to-noise-ratio metric 420, as shown in FIG. 4, the intensity correction and quality calibration system 106 generates a noise component from the plurality of intensity correction parameters 412 and the one or more variation correction coefficients 414 to determine the signal-to-noise-ratio metric 420 directly from outputs of the maximum likelihood estimation model 410.

In one or more embodiments, for example, the intensity correction and quality calibration system 106 determines a noise level component 418 (sse, i.e., “Sum of Squared Error”) of the signal-to-noise-ratio metric 420 as follows:

$$sse = {( 1 - \hat{a} )}^{2} \overline{xx} + {\hat{d}}_{1}^{2} + {\hat{d}}_{2}^{2} + \overline{ee} + 2 ( 1 - \hat{a} ) ( \overline{xe} + {\hat{d}}_{1} {\overline{x}}_{1} - {\hat{d}}_{2} {\overline{x}}_{2} ) - 2 ( {\hat{d}}_{1} {\overline{e}}_{1} - {\hat{d}}_{2} {\overline{e}}_{2} )$$

where e̅e̅ represents a mean squared sum of a first intensity error estimation ē₁ and a second intensity error estimation ē₂ of the plurality of intensity correction parameters 412, such that

$$\overline{ee} {=}^{△} \frac{1}{C} {\sum }_{c = 1}^{C} ( {e}_{c , 1}^{2} + {e}_{c , 2}^{2} ) .$$

Accordingly, in one or more embodiments, the intensity correction and quality calibration system 106 utilizes the mean squared sum e̅e̅, in addition to the plurality of intensity correction parameters 412 (\hat{x}₁, \hat{x}₂, x̅x̅, ê₁, ê₂, and x̅e̅) and the one or more variation correction coefficients 414 (â, d₁, and d₂) generated by the maximum likelihood estimation model 410, to determine the noise level component 418 (sse) of the signal-to-noise-ratio metric 420. In some embodiments, for example, the intensity correction and quality calibration system 106 determines the signal-to-noise-ratio metric 420 (snr) as follows:

$$snr = \frac{{\hat{a}}^{2}}{sse} \times {μ}_{c}$$

where μ_{c} is a normalization factor.

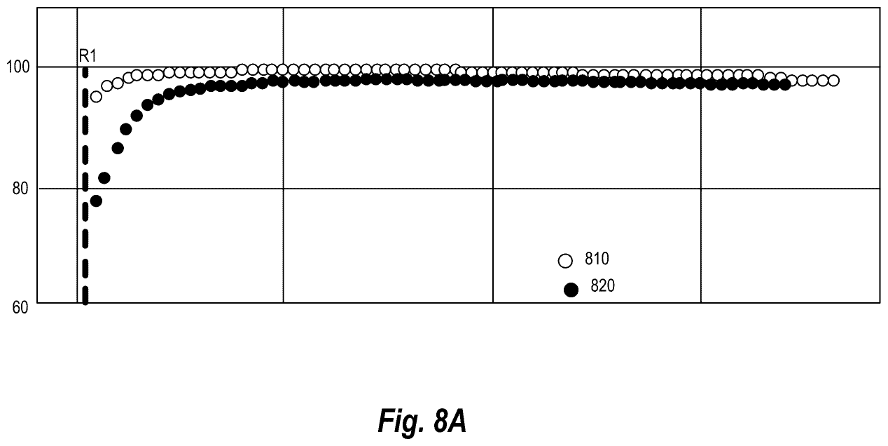

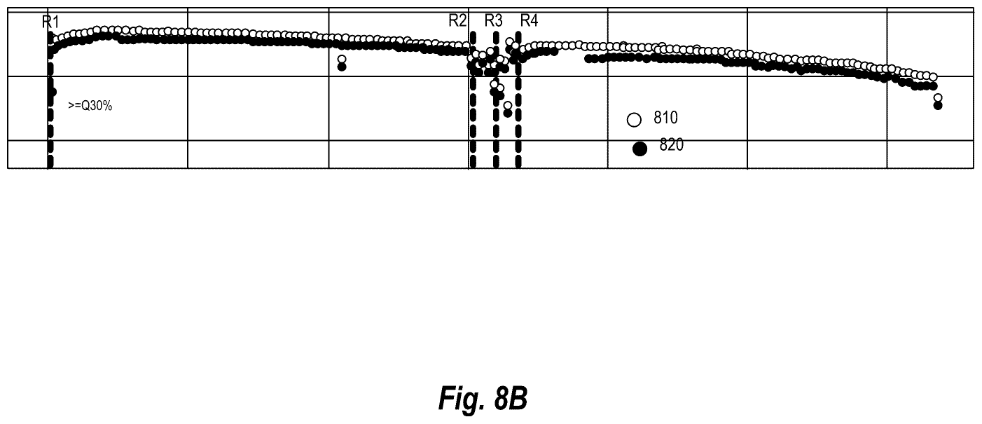

Accordingly, as shown in FIG. 4, the intensity correction and quality calibration system 106 determines the signal-to-noise-ratio metric 420 (snr) based on the scaling factor 416 (â) and the noise level component 418 (sse) derived directly from terms determined per the maximum likelihood estimation model 410 approaching the least squares solution 408 for the plurality of intensity correction parameters 412 and the one or more variation correction coefficients 414 corresponding to the signal 404. Indeed, as mentioned above, in one or more embodiments, the intensity correction and quality calibration system 106 implements an improved method for determining signal-to-noise-ratio metrics across successive sequencing cycles, thus providing for accelerated convergence, relative to existing systems, of signal-to-noise-ratio metrics during early sequencing cycles of a sequencing run (e.g., as further illustrated by FIGS. 8A-8B).

To further illustrate, in contrast to the methods disclosed herein, many existing sequencing systems that utilize a signal-to-noise-ratio metric rely upon a rolling average of corrected intensity values to estimate or determine at least part (e.g., the noise component) of the signal-to-noise-ratio metric. Thus, by utilizing the mean squared sum e as described above to derive the noise level component 418 directly from the plurality of intensity correction parameters 412 and the one or more variation correction coefficients 414, the intensity correction and quality calibration system 106 determines a signal-to-noise-ratio ratio for each successive sequencing cycle of a sequencing run without influence from previous sequencing cycles. Consequently, the intensity correction and quality calibration system 106 averts any detrimental effects of errors in previous sequencing cycles, resulting in accelerated convergence of signal-to-noise-ratio metric and quality metric values and improved accuracy across sequencing cycles of a sequencing run. Furthermore, the foregoing method for signal-to-noise-ratio metric determination does not require additional memory or computation resources to implement, as the parameters from which the intensity correction and quality calibration system 106 derives the signal-to-noise-ratio metric are already generated for the purposes of intensity value correction and optimization. Additionally, the intensity correction and quality calibration system uses the memory space relied on by old systems for those values determined outside the channel estimations to store the new mean squared sum values that enable direct determination of the noise component.

As previously mentioned, in some embodiments, the intensity correction and quality calibration system 106 determines a quality metric estimating an error of a nucleobase call generated for a signal in a series of signals corresponding to a series of sequencing cycles. For example, FIG. 5 illustrates the intensity correction and quality calibration system 106 generating a quality metric 520 for a nucleobase call 510 based on a signal-to-noise-ratio metric 512 generated for a signal associated with a given sequencing cycle of a series of sequencing cycles.

As shown in FIG. 5, the intensity correction and quality calibration system 106 determines the signal-to-noise-ratio metric 512 corresponding to a signal captured with an image 502 (or multiple images) and generates a nucleobase call 510 for the signal. As shown, the intensity correction and quality calibration system 106 determines intensity values 504 for the signal associated with the image 502, determines a plurality of intensity correction parameters 506 for the signal (e.g., according to a least squares solution, such as described above in relation to FIG. 4), and utilizes the plurality of intensity correction parameters 506 to generate corrected intensity values 508 for the signal to determine the nucleobase call 510.

As further shown in FIG. 5, the intensity correction and quality calibration system 106 generates the signal-to-noise-ratio metric 512 based on the plurality of intensity correction parameters 506 (e.g., as described above in relation to FIG. 4). In addition, as shown in FIG. 5, the intensity correction and quality calibration system 106 generates the quality metric 520 for the nucleobase call 510 based on the signal-to-noise-ratio metric 512. In particular, the intensity correction and quality calibration system 106 generates the quality metric 520 utilizing a base-call-quality model 514. In one or more embodiments, the base-call-quality model 514 accepts one or more dimensions (e.g., inputs) related to features of a signal and/or features of the corresponding section of a nucleotide-sample slide and generates the quality metric 520 based on those dimensions. Accordingly, the intensity correction and quality calibration system 106 can provide the signal-to-noise-ratio metric 512 as an input to the base-call-quality model 514.

As also shown in FIG. 5, in some embodiments, the base-call-quality model 514 includes a Phred algorithm (as indicated by the graph 516a). Indeed, in some embodiments, the intensity correction and quality calibration system 106 uses the Phred algorithm to determine the quality metric 520. For instance, in some implementations, the intensity correction and quality calibration system 106 uses the Phred algorithm to generate and/or calibrate a quality reference table for use in determining quality scores as further described below in relation to FIG. 6. Indeed, as also shown in FIG. 5, the base-call-quality model 514 can include a quality reference table 516b comprising multiple values or ranges of quality metrics associated with a plurality of quality predictor values, such as various values or ranges of signal-to-noise-ratio metrics generated by the intensity correction and quality calibration system 106 during sequencing of a dataset comprising multiple well-characterized genomic samples. Thus, in certain embodiments, the intensity correction and quality calibration system 106 utilizes the quality reference table 516b of the base-call-quality model 514 to generate the quality metric 520 for the signal from the image 502. Thus, in one or more embodiments, the intensity correction and quality calibration system 106 uses the Phred algorithm to generate a Phred quality score (e.g., a Q-score) that estimates the accuracy of the nucleobase call. In other words, in some cases, the quality metric 520 includes a Phred quality score generated by the Phred algorithm of the base-call-quality model 514, such as the Phred algorithm and associated Phred quality scores described by Method and System for Determining the Accuracy of DNA Base Identifications, U.S. Pat. No. 8,392,126 (filed Sep. 23, 2009), which is incorporated herein by reference in its entirety.

Also, as shown in FIG. 5, the base-call-quality model 514 can utilize one or more chastity values 518 in addition to the signal-to-noise-ratio metric 512 to determine the quality metric 520 for the nucleobase call 510. As mentioned above, in some embodiments, the one or more chastity values 518 can be determined for a corresponding signal (e.g., related to the image 502) as the ratio of a distance between the intensity associated with the signal and the nearest nucleobase centroid to ta distance between the intensity and another centroid (e.g., the second nearest centroid).

In some cases, the intensity correction and quality calibration system 106 further utilizes the quality metric 520 determined for the nucleobase call 510 to map the nucleobase call to a reference genome and perform variant calling. In particular, the intensity correction and quality calibration system 106 can map the oligonucleotide located at the section of the nucleotide-sample slide emitting the signal to a reference genome and perform variant calling. Accordingly, in one or more embodiments, the intensity correction and quality calibration system 106 detects a signal by detecting the signal from labeled nucleotide bases incorporated into a growing oligonucleotide at a genomic position later determined in alignment with a reference genome. Additionally, the intensity correction and quality calibration system 106 generates the signal-to-noise-ratio metric 512 for the nucleobase call 510 at the genomic position corresponding to the signal. Further, the intensity correction and quality calibration system 106 can determine the quality metric 520 for the nucleobase call 510 and utilize the quality metric 520 to map the nucleobase call 510 to the reference genome and perform variant calling.

As previously mentioned, in some embodiments, the intensity correction and quality calibration system 106 utilizes one or more signal-to-noise-ratio metrics generated according to the foregoing methods to generate and/or calibrate a quality reference table correlating a distribution of quality predictor values with a plurality of quality metrics. For example, FIG. 6 illustrates the intensity correction and quality calibration system 106 generating signal-to-noise-ratio metrics 608 for one or more reference genome samples 602 and generating or calibrating a quality reference table 614 based on a set of quality predictor values 606 (including the signal-to-noise-ratio metrics 608) corresponding to respective ones of the one or more reference genome samples 602.

As shown in FIG. 6, the intensity correction and quality calibration system 106 generates or identifies (or receives) nucleobase-call data 604 for the one or more reference genome samples 602. In one or more embodiments, for example, the one or more reference genome samples 602 comprise well-characterized genomic samples for which ground truth data (e.g., proven or otherwise reliable nucleobase calls and related information) is available. Further, the intensity correction and quality calibration system 106 extracts or otherwise determines the set of quality predictor values 606 from the nucleobase-call data 604, including the signal-to-noise-ratio metrics 608 corresponding to nucleobase calls of the one or more reference genome samples 602. In addition to the signal-to-noise-ratio metrics 608, the set of quality predictor values 606 can include various metrics related to base-call quality, such as but not limited to chastity values, intensity values, and so forth.

As also shown in FIG. 6, the intensity correction and quality calibration system 106 utilizes a base-call-quality model 610 to determine the quality metrics 612 based on the signal-to-noise-ratio metrics 608 (and other quality predictor metrics) corresponding to the nucleobase-call data 604 (e.g., as described above in relation to FIG. 5). Further, in some implementations, the intensity correction and quality calibration system 106 utilizes the base-call-quality model 610 to generate or calibrate (e.g., modify) the quality reference table 614 to reflect relationships between the set of quality predictor values 606 and the quality metrics 612 (e.g., for determining base-call quality in subsequent analyses, such as described above in relation to FIG. 5). To illustrate, in some cases, the intensity correction and quality calibration system 106 uses a Phred algorithm of the base-call-quality model 610 to generate or calibrate the quality reference table 614. The quality reference table 614, for example, can include a statistical representation of quality metrics (including the quality metrics 612) derived from various well-characterized genomic samples (e.g., the one or more reference genome samples 602) sequenced and evaluated according to the foregoing methods to provide a quality reference table 614 for subsequent sequencing applications. Alternatively or additionally, the intensity correction and quality calibration system 106 can utilize signal-to-noise-ratio metrics generated for such well-characterized samples to train a base-call-quality model (such as the base-call-quality model 610) for use in subsequent sequencing applications.

Accordingly, by deriving signal-to-noise-ratio metrics for sequencing signals according to the disclosed methods, the intensity correction and quality calibration system 106 can generate a quality reference table (or calibrate an existing quality reference table) with increased accuracy over existing sequencing systems that suffer from poor convergence and inaccuracies in determining signal-to-noise-ratio metrics for earlier sequencing cycles in sequencing runs. Indeed, by determining improved signal-to-noise-ratio metrics, the intensity correction and quality calibration system 106 can generate quality reference tables that more accurately correlate such signal-to-noise-ratio metrics with corresponding quality metrics. Consequently, the intensity correction and quality calibration system 106 can provide a quality reference table which provides for increased accuracy and reliability in determining quality metrics for nucleobase calls based on quality predictor values for a given sequencing signal.

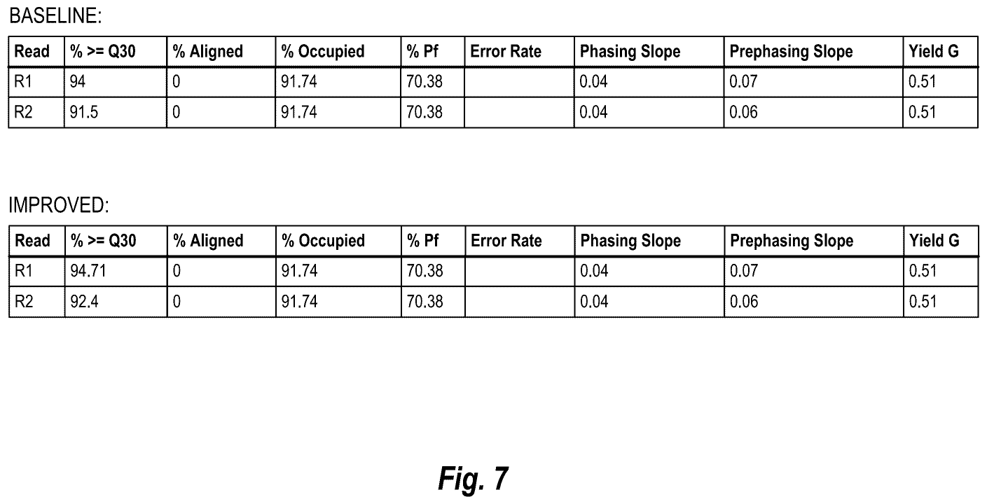

As mentioned above, in certain described embodiments, the intensity correction and quality calibration system 106 implements nucleotide-base calling with increased accuracy relative to existing sequencing systems. To illustrate, FIG. 7 shows experimental results of the intensity correction and quality calibration system 106 generating nucleobase calls with increased accuracy over an existing sequencing system. Specifically, FIG. 7 includes a table of experimental results of determining nucleobase calls with quality metrics generated by an existing sequencing system (“BASELINE”) and by the intensity correction and quality calibration system 106 using the improved signal-to-noise-ratio metric (“IMPROVED”). The BASELINE system determines the nucleobase calls and quality metrics using the indirect approach to determining signal-to-noise. Indeed, as shown in FIG. 7, nucleobase calls generated by the intensity correction and quality calibration system 106 exhibit increased overall accuracy compared to existing sequencing systems, such as indicated by the relative increase in quality metrics (Q-scores) above Q30 between the existing sequencing system and the intensity correction and quality calibration system 106. Notably, these results were obtained without recalibration of the quality reference table that was used. Recalibration of the quality reference table would likely further improve the results obtained by the intensity correction and quality calibration system 106.

Moreover, as also mentioned above, in certain described embodiments, the intensity correction and quality calibration system 106 provides for accelerated convergence of signal-to-noise-ratio metrics during early sequencing cycles, resulting in improved overall accuracy of determining quality metrics over a series of sequencing cycles. To illustrate, FIGS. 8A-8B show experimental results of the intensity correction and quality calibration system 106 generating signal-to-noise-ratio metrics for signals from multiple series of sequencing cycles according to one or more embodiments. In particular, FIGS. 8A-8B illustrate comparative results of determining signal-to-noise-ratio metrics and quality metrics according to one or more embodiments.

Specifically, FIG. 8A includes a graph representation of signal-to-noise-ratio metrics generated for a sequencing run (R1) utilizing the intensity correction and quality calibration system 106 (results 810) and an existing sequencing system (results 820). Indeed, as FIG. 8A illustrates, the intensity correction and quality calibration system 106 exhibits accelerated convergence of signal-to-noise-ratio metrics during early cycles of the example sequencing run, relative to the existing sequencing system.

Relatedly, FIG. 8B, includes a graph representation of quality metrics generated across subsequent sequencing runs (R1-R4) utilizing signal-to-noise-ratio metrics generated by the intensity correction and quality calibration system 106 (results 810) and the existing sequencing system (results 820). Indeed, as FIG. 8B illustrates, the aforementioned accelerated convergence of signal-to-noise-ratio metrics generated by the intensity correction and quality calibration system 106 results in improved overall accuracy of determining quality metrics over multiple sequencing cycles, particularly in early sequencing cycles of a sequencing run.

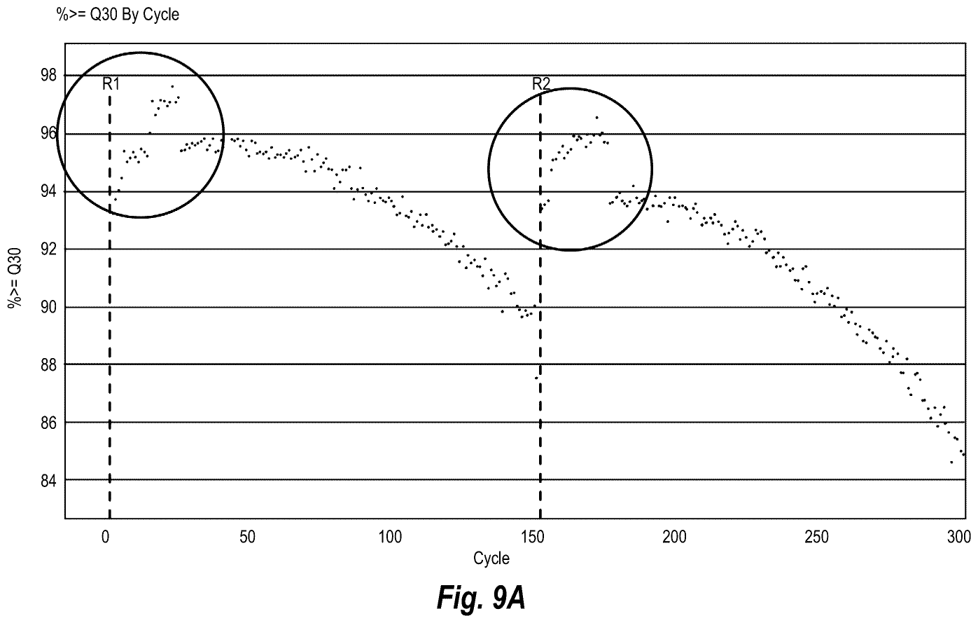

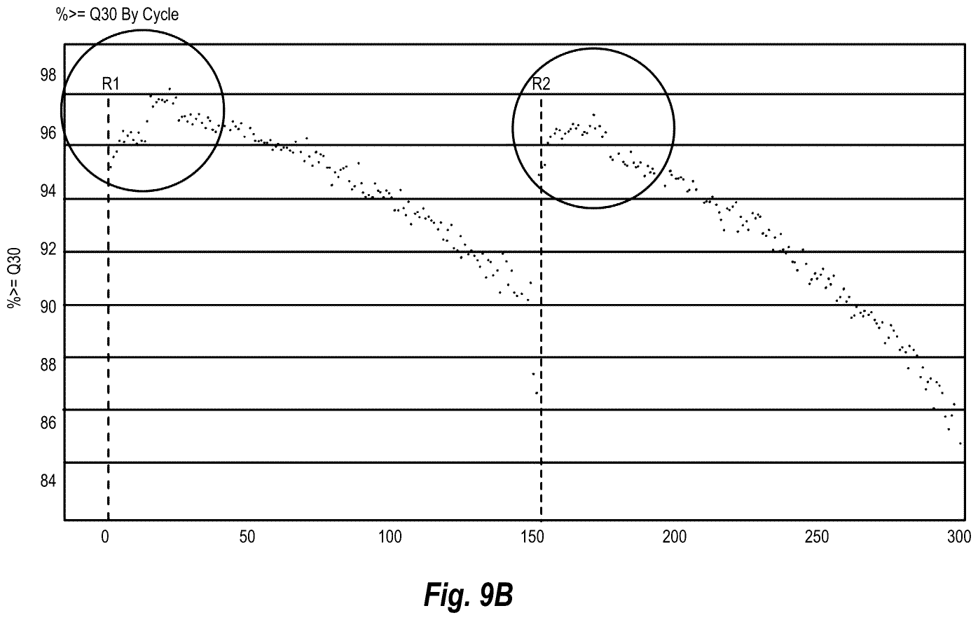

Moreover, FIGS. 9A-9B include additional illustrations of experimental results of the intensity correction and quality calibration system 106 determining quality metrics for nucleobase calls utilizing signal-to-noise-ratio metrics generated according to one or more embodiments. Specifically, FIG. 9A includes a graphical representation of quality metrics generated by an existing sequencing system, whereas FIG. 9B includes a graphical representation of quality metrics generated by the intensity correction and quality calibration system 106 according to one or more embodiments. Indeed, as indicated in FIGS. 9A-9B, the intensity correction and quality calibration system 106 can generate quality metrics for sequencing cycles with increased overall accuracy compared to existing systems. In particular, while FIG. 9A shows a relatively drastic decrease in the quality metrics at or near Cycle 25 in R1 or R2, FIG. 9B (quality metrics generated using the intensity correction and quality calibration system 106) portrays a more consistent (e.g., smoother) distribution of quality metrics compared to that of FIG. 9A (quality metrics generated using an existing sequencing system).

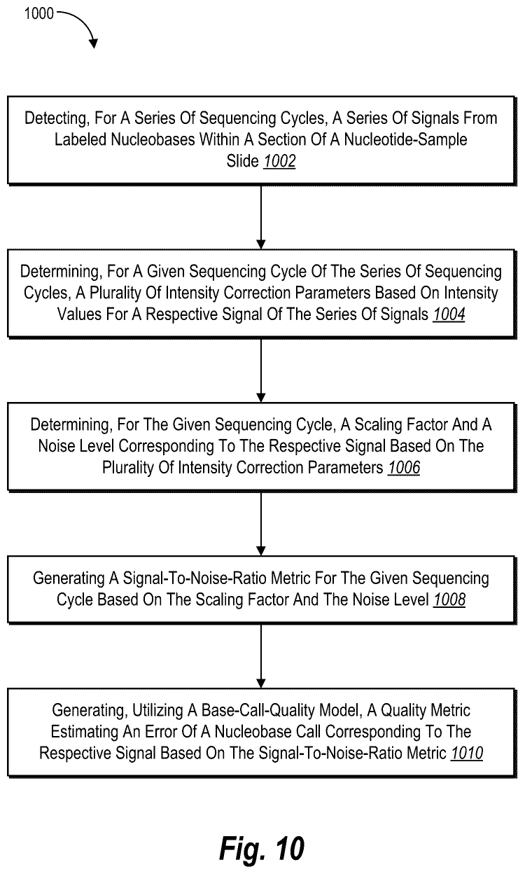

Turning now to FIG. 10, this figure illustrates an example flowchart of a series of acts for generating and utilizing a signal-to-noise-ratio metric for a signal corresponding to a given sequencing cycle in accordance with one or more embodiments. While FIG. 10 illustrates acts according to particular embodiments, alternative embodiments may omit, add to, reorder, and/or modify any of the acts shown in FIG. 10. The acts of FIG. 10 can be performed as part of a method. Alternatively, a non-transitory computer readable storage medium can comprise instructions that, when executed by one or more processors, cause a computing device to perform the acts depicted in FIG. 10. In still further embodiments, a system comprising at least one processor and a non-transitory computer readable medium comprising instructions that, when executed by one or more processors, cause the system to perform the acts of FIG. 10.

As shown in FIG. 10, the series of acts 1000 includes an act 1002 of detecting, for a series of sequencing cycles, a series of signals from labeled nucleobases within a section of a nucleotide-sample slide, an act 1004 of determining, for a given series of sequencing cycles, a plurality of intensity correction parameters based on intensity values for a respective series of signals, an act 1006 of determining, for the given sequencing cycle, a scaling factor and a noise level corresponding to the respective signal based on the plurality of intensity correction parameters, an act 1008 of generating a signal-to-noise-ratio metric for the given sequencing cycle based on the scaling factor and the noise level, and an act 1010 of generating, utilizing a base-call-quality model, a quality metric estimating an error of a nucleobase call corresponding to the respective signal based on the signal-to-noise-ratio metric.

For example, the series of acts 1000 can include acts to perform any of the operations described in the following clauses:

CLAUSE 1. A method comprising:
detecting, for a series of sequencing cycles, a series of signals from labeled nucleotide bases within a section of a nucleotide-sample slide;
determining, for the series of sequencing cycles, a plurality of intensity correction parameters based on respective intensity values of the series of signals;
determining, for a given sequencing cycle, a scaling factor and a noise level corresponding to a respective signal based on respective intensity correction parameters for the given sequencing cycle;
generating a signal-to-noise-ratio metric for the given sequencing cycle based on the scaling factor and the noise level; and
generating, utilizing a base-call-quality model, a quality metric estimating an error of a nucleotide-base call corresponding to the respective signal based on the signal-to-noise-ratio metric.

CLAUSE 2. The method of clause 1, further comprising determining the plurality of intensity correction parameters utilizing a maximum likelihood estimation model to predict variation correction coefficients corresponding to the respective signal.

CLAUSE 3. The method of clause 2, wherein the predicted variation correction coefficients comprise the scaling factor for the given sequencing cycle and one or more correction offset factors for respective channels of the respective signal.

CLAUSE 4. The method of any of clauses 2-3, further comprising configuring the maximum likelihood estimation model to approach a least squares solution for the variation correction coefficients corresponding to the respective signal.

CLAUSE 5. The method of any of clauses 1-4, further comprising determining the noise level corresponding to the respective signal based on a mean squared sum of two intensity correction parameters of the respective intensity correction parameters for the given sequencing cycle.

CLAUSE 6. The method of clause 5, wherein the two intensity correction parameters comprise a first intensity error estimation for a first intensity channel of the respective signal for the given sequencing cycle and a second intensity error estimation for a second intensity channel of the respective signal.

CLAUSE 7. The method of any of clauses 1-6, further comprising determining the noise level for the given sequencing cycle without correlation to intensity correction parameters corresponding to other sequencing cycles of the series of sequencing cycles.

CLAUSE 8. The method of any of clauses 1-7, further comprising generating, utilizing the base-call-quality model, the quality metric based on the signal-to-noise-ratio metric and further based on one or more chastity values corresponding to the respective signal.

CLAUSE 9. The method of any of clauses 1-8, further comprising generating the quality metric based on the signal-to-noise-ratio metric by generating a Phred quality score estimating an accuracy of the nucleotide-base call corresponding to the respective signal based on the signal-to-noise-ratio metric.

CLAUSE 10. The method of any of clauses 1-9, wherein the section of the nucleotide-sample slide corresponds to an individual cluster of oligonucleotides within the nucleotide-sample slide.

CLAUSE 11. The method of any of clauses 1-10, further comprising generating additional signal-to-noise-ratio metrics for additional sequencing cycles of the series of sequencing cycles based on respective additional signals of the series of signals from the labeled nucleotide bases within the section of the nucleotide-sample slide.

CLAUSE 12. The method of clause 11, further comprising generating, utilizing the base-call-quality model, additional quality metrics estimating respective errors of additional nucleotide-base calls corresponding to the respective additional signals based on the additional signal-to-noise-ratio metrics for the additional sequencing cycles.

CLAUSE 13. The method of clause 12, further comprising generating, based at least in part on the signal-to-noise-ratio metric and the additional signal-to-noise-ratio metrics, a quality reference table correlating a distribution of quality predictor values with a plurality of quality metrics.

CLAUSE 14. The method of any of clauses 12-13, further comprising modifying one or more quality metrics associated with one or more quality predictor values within a quality reference table.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using T-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially, and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the intensity correction and quality calibration system 106 can include software, hardware, or both. For example, the components of the intensity correction and quality calibration system 106 can include one or more instructions stored on a computer-readable storage medium and executable by processors of one or more computing devices (e.g., the client device 114, the local device 108, or the server device(s) 110). When executed by the one or more processors, the computer-executable instructions of the intensity correction and quality calibration system 106 can cause the computing devices to perform the bubble detection methods described herein. Alternatively, the components of the intensity correction and quality calibration system 106 can comprise hardware, such as special purpose processing devices to perform a certain function or group of functions. Additionally, or alternatively, the components of the intensity correction and quality calibration system 106 can include a combination of computer-executable instructions and hardware.

Furthermore, the components of the intensity correction and quality calibration system 106 performing the functions described herein with respect to the intensity correction and quality calibration system 106 may, for example, be implemented as part of a stand-alone application, as a module of an application, as a plug-in for applications, as a library function or functions that may be called by other applications, and/or as a cloud-computing model. Thus, components of the intensity correction and quality calibration system 106 may be implemented as part of a stand-alone application on a personal computing device or a mobile device. Additionally, or alternatively, the components of the intensity correction and quality calibration system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 11 illustrates a block diagram of a computing device 1100 that may be configured to perform one or more of the processes described above. One will appreciate that one or more computing devices such as the computing device 1100 may implement the intensity correction and quality calibration system 106 and the sequencing device system 104. As shown by FIG. 11, the computing device 1100 can comprise a processor 1102, a memory 1104, a storage device 1106, an I/O interface 1108, and a communication interface 1110, which may be communicatively coupled by way of a communication infrastructure 1112. In certain embodiments, the computing device 1100 can include fewer or more components than those shown in FIG. 11. The following paragraphs describe components of the computing device 1100 shown in FIG. 11 in additional detail.

In one or more embodiments, the processor 1102 includes hardware for executing instructions, such as those making up a computer program. As an example, and not by way of limitation, to execute instructions for dynamically modifying workflows, the processor 1102 may retrieve (or fetch) the instructions from an internal register, an internal cache, the memory 1104, or the storage device 1106 and decode and execute them. The memory 1104 may be a volatile or non-volatile memory used for storing data, metadata, and programs for execution by the processor(s). The storage device 1106 includes storage, such as a hard disk, flash disk drive, or other digital storage device, for storing data or instructions for performing the methods described herein.

The I/O interface 1108 allows a user to provide input to, receive output from, and otherwise transfer data to and receive data from computing device 1100. The I/O interface 1108 may include a mouse, a keypad or a keyboard, a touch screen, a camera, an optical scanner, network interface, modem, other known I/O devices or a combination of such I/O interfaces. The I/O interface 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1108 is configured to provide graphical data to a display for presentation to a user. The graphical data may be representative of one or more graphical user interfaces and/or any other graphical content as may serve a particular implementation.

The communication interface 1110 can include hardware, software, or both. In any event, the communication interface 1110 can provide one or more interfaces for communication (such as, for example, packet-based communication) between the computing device 1100 and one or more other computing devices or networks. As an example, and not by way of limitation, the communication interface 1110 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1110 may facilitate communications with various types of wired or wireless networks. The communication interface 1110 may also facilitate communications using various communication protocols. The communication infrastructure 1112 may also include hardware, software, or both that couples components of the computing device 1100 to each other. For example, the communication interface 1110 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer-readable medium storing instructions that, when executed by the at least one processor, cause the system to:
detect, for a series of sequencing cycles, a series of signals from labeled nucleotide bases within a section of a nucleotide-sample slide;
determine, for the series of sequencing cycles, a plurality of intensity correction parameters based on respective intensity values of the series of signals;
determine, for a given sequencing cycle, a scaling factor and a noise level corresponding to a respective signal based on respective intensity correction parameters for the given sequencing cycle;
generate a signal-to-noise-ratio metric for the given sequencing cycle based on the scaling factor and the noise level; and
generate, utilizing a base-call-quality model, a quality metric estimating an error of a nucleotide-base call corresponding to the respective signal based on the signal-to-noise-ratio metric.

2. The system of claim 1, further comprising instructions that, when executed by the at least one processor, cause the system to determine the plurality of intensity correction parameters utilizing a maximum likelihood estimation model to predict variation correction coefficients corresponding to the respective signal.

3. The system of claim 2, wherein the predicted variation correction coefficients comprise the scaling factor for the given sequencing cycle and one or more correction offset factors for respective channels of the respective signal.

4. The system of claim 2, further comprising instructions that, when executed by the at least one processor, cause the system to configure the maximum likelihood estimation model to approach a least squares solution for the variation correction coefficients corresponding to the respective signal.

5. The system of claim 1, further comprising instructions that, when executed by the at least one processor, cause the system to determine the noise level corresponding to the respective signal based on a mean squared sum of two intensity correction parameters of the respective intensity correction parameters for the given sequencing cycle.

6. The system of claim 5, wherein the two intensity correction parameters comprise a first intensity error estimation for a first intensity channel of the respective signal for the given sequencing cycle and a second intensity error estimation for a second intensity channel of the respective signal.

7. The system of claim 1, further comprising instructions that, when executed by the at least one processor, cause the system to determine the noise level for the given sequencing cycle without correlation to intensity correction parameters corresponding to other sequencing cycles of the series of sequencing cycles.

8. The system of claim 1, further comprising instructions that, when executed by the at least one processor, cause the system to generate, utilizing the base-call-quality model, the quality metric based on the signal-to-noise-ratio metric and further based on one or more chastity values corresponding to the respective signal.

9. The system of claim 1, further comprising instructions that, when executed by the at least one processor, cause the system to generate the quality metric based on the signal-to-noise-ratio metric by generating a Phred quality score estimating an accuracy of the nucleotide-base call corresponding to the respective signal based on the signal-to-noise-ratio metric.

10. The system of claim 1, wherein the section of the nucleotide-sample slide corresponds to an individual cluster of oligonucleotides within the nucleotide-sample slide.

11. The system of claim 1, further comprising instructions that, when executed by the at least one processor, cause the system to generate additional signal-to-noise-ratio metrics for additional sequencing cycles of the series of sequencing cycles based on respective additional signals of the series of signals from the labeled nucleotide bases within the section of the nucleotide-sample slide.

12. The system of claim 11, further comprising instructions that, when executed by the at least one processor, cause the system to generate, utilizing the base-call-quality model, additional quality metrics estimating respective errors of additional nucleotide-base calls corresponding to the respective additional signals based on the additional signal-to-noise-ratio metrics for the additional sequencing cycles.

13. The system of claim 12, further comprising instructions that, when executed by the at least one processor, cause the system to generate, based at least in part on the signal-to-noise-ratio metric and the additional signal-to-noise-ratio metrics, a quality reference table correlating a distribution of quality predictor values with a plurality of quality metrics.

14. The system of claim 12, further comprising instructions that, when executed by the at least one processor, cause the system to modify one or more quality metrics associated with one or more quality predictor values within a quality reference table.

15. A non-transitory computer readable medium storing instructions that, when executed by at least one processor, cause a computing device to:
detect, for a series of sequencing cycles, a series of signals from labeled nucleotide bases within a section of a nucleotide-sample slide;
determine, for the series of sequencing cycles, a plurality of intensity correction parameters based on respective intensity values of the series of signals;
determine, for a given sequencing cycle, a scaling factor and a noise level corresponding to a respective signal based on respective intensity correction parameters for the given sequencing cycle;
generate a signal-to-noise-ratio metric for the given sequencing cycle based on the scaling factor and the noise level; and
generate, utilizing a base-call-quality model, a quality metric estimating an error of a nucleotide-base call corresponding to the respective signal based on the signal-to-noise-ratio metric.

16. The non-transitory computer readable medium of claim 15, further comprising instructions that, when executed by the at least one processor, cause the computing device to determine the plurality of intensity correction parameters utilizing a maximum likelihood estimation model to predict variation correction coefficients corresponding to the respective signal.

17. The non-transitory computer readable medium of claim 16, wherein the predicted variation correction coefficients comprise the scaling factor for the given sequencing cycle and one or more correction offset factors for respective channels of the respective signal.

18. The non-transitory computer readable medium of claim 16, further comprising instructions that, when executed by the at least one processor, cause the computing device to configure the maximum likelihood estimation model to approach a least squares solution for the variation correction coefficients corresponding to the respective signal.

19. The non-transitory computer readable medium of claim 15, further comprising instructions that, when executed by the at least one processor, cause the computing device to determine the noise level for the given sequencing cycle without correlation to intensity correction parameters corresponding to other sequencing cycles of the series of sequencing cycles.

20. A computer-implemented method comprising:
detecting, for a series of sequencing cycles, a series of signals from labeled nucleotide bases within a section of a nucleotide-sample slide;
determining, for the series of sequencing cycles, a plurality of intensity correction parameters based on respective intensity values of the series of signals;
determining, for a given sequencing cycle, a scaling factor and a noise level corresponding to a respective signal based on respective intensity correction parameters for the given sequencing cycle;
generating a signal-to-noise-ratio metric for the given sequencing cycle based on the scaling factor and the noise level; and
generating, utilizing a base-call-quality model, a quality metric estimating an error of a nucleotide-base call corresponding to the respective signal based on the signal-to-noise-ratio metric.
